(19) United States
(12) Patent Application Publication
(10) Pub. No.: US 2019/0255056 A1
(43) Pub. Date: Aug. 22, 2019

(54) A HCK INHIBITOR AND A BCL-2 INHIBITOR FOR TREATING ACUTE MYELOID LEUKEMIA

(71) Applicant: RIKEN, Wako-shi, Saitama (JP)

(72) Inventors: Fumihiko ISHIKAWA, Saitama (JP); Yoriko SAITO, Saitama (JP)

(73) Assignee: RIKEN, Wako-shi, Saitama (JP)

(21) Appl. No.: 16/333,995

(22) PCT Filed: Sep. 15, 2017
(86) PCT No.: PCT/JP2017/033482

§ 371 Date: Mar. 15, 2019

Related U.S. Application Data
(60) Provisional application No. 62/394,871, filed on Sep. 15, 2016.

(51) Int. Cl. A61K 31/519; A61K 31/4709; A61K 31/437; A61P 35/02

(52) U.S. Cl. CPC A61K 31/519; A61K 31/4709; A61K 45/06; A61P 35/02; A61K 31/437

(57) ABSTRACT

This invention relates to compounds, compositions and methods for treating leukemia, such as acute myeloid leukemia, in subjects where one or more mutations in FLT3 kinase are present.

### TECHNICAL FIELD

The present invention relates to compounds, compositions and methods for treating leukemia, such as acute myeloid leukemia.

### BACKGROUND ART

Over the last decade, leukemia stem cells (LSCs) have become recognized as key players in human acute myeloid leukeumia (AML) pathogenesis as well as chemotherapy resistance and relapse. The Src family kinase (SFK) hematopoietic cell kinase, or HCK, is overrepresented in primary human AML LSCs in comparison to normal hematopoietic stem cells (HSCs). The importance of HCK and other SFKs, including LYN and FGR, in myeloid proliferation and differentiation has been demonstrated in knockout mice. The myeloid-specific SFKs participate in wild-type and mutant kinase receptor (KIT) and fms-like tyrosine kinase 3 (FLT3) signaling and in the activation of the signal transducer and activator of transcription 5 (STAT5). Myeloid-specific SFKs also are involved in extracellular signal-regulated kinase (ERK) pathways downstream of breakpoint cluster gene-Abelson murine leukemia fusion protein (BCR−ABL), and are involved in leukemogenesis in a mouse model of BCR−ABL+B−ALL (acute lymphoblastic leukemia).

FLT3 is a type III receptor tyrosine kinase that plays important roles in the differentiation and survival of hematopoietic stem cells in bone marrow and has been observed to be over-expressed in AML and ALL. A variety of gain-of-function mutations have been identified in these AML patients, such as FLT3−ITD (internal tandem duplication), FLT3−D835Y/E/V/H, and FLT3−K663Q, among which FLT3−ITD accounts for about 30% of AML occurrence and is associated with poor prognosis. The mutated FLT3−ITD kinase promotes AML blast survival and proliferation through the downstream signaling mediators, including StatS, ERK and AKT. Therefore, FLT3−ITD kinase has been considered as a validated drug discovery target for FLT3−ITD positive AML.

A number of small molecule inhibitors have been reported to exhibit potent FLT3 kinase inhibitory activities such as sunitinib, sorafenib, PKC412, CEP-701, UNC2025, MLN518, KW-2449 and AMG-925. In addition, the relatively more selective second generation FLT3 inhibitors such as AC2206, crenolanib and PLX3397 are being clinically evaluated and have shown initial transient responses, but usually followed by quick development of resistance.

Mutations in genes such as NPM1, TET2, WT1, IDH1/2 and DNMT3A are commonly found in AML, and recent work utilizing next-generation sequencing (NGS) suggests that certain mutations occur earlier than others based on variant allele frequencies (VAFs). Pre-leukemic cells harboring early somatic mutations are thought to contribute to leukemogenesis and disease relapse. However, these mutations may not be sufficient for leukemogenesis nor identify cells destined to become malignant.

Thus, there exists a need for elucidation of causational mutations for AML and effective treatments based on inhibiting or blocking aberrant pathways generated by those mutations. One effective combination that can provide durable remission of AML is provided in the combination of a dual HCK/FLT3−ITD inhibitor and a BCL-2 inhibitor.

### SUMMARY OF INVENTION

The present invention is summarized as follows.

1. A method of co-inhibiting HCK and BCL-2 in a cell, comprising contacting the cell with an HCK inhibitor and a BCL-2 inhibitor, or a pharmaceutically acceptable composition thereof.

2. A method of killing a cell having an FLT3−ITD mutation, comprising contacting the cell with an HCK inhibitor and a BCL-2 inhibitor, or a pharmaceutically acceptable composition thereof.

3. The method of item 1 or 2, further comprising contacting the cell with an FLT3−ITD inhibitor.

4. The method of item 1 or 2, wherein the HCK inhibitor is a dual HCK/FLT3−ITD inhibitor.

5. A method of treating acute myeloid leukemia, comprising conjointly administering to a subject an HCK inhibitor and a BCL-2 inhibitor, or a pharmaceutically acceptable composition thereof.

6. The method of item 5, wherein the subject has FLT3−ITD+ acute myeloid leukemia.

7. The method of item 5 or 6, wherein the subject has malignant hematopoiesis and/or non-malignant multilineage hematopoiesis characterized by cells having one or more mutations in a gene selected from DNMT3A, IDH2, IDH1, NPM1, TET2, CEBPA, ASXL1, EZH2, SETBP1, SMC3, KIT, NRAS, and WT1.

8. The method of any one of items 5-7, further comprising conjointly administering a FLT3−ITD inhibitor.

9. The method of any one of items 5-8, wherein the HCK inhibitor is a dual HCK/FLT3−ITD inhibitor.

10. The method of item 8 or 9, wherein the HCK inhibitor, the FLT3−ITD inhibitor, and the BCL-2 inhibitor are administered simultaneously or sequentially in separate unit dosage forms.

11. The method of any one of items 5-10, comprising administering a single unit dosage form comprising an HCK inhibitor, a BCL-2 inhibitor, and a pharmaceutically acceptable carrier, adjuvant, or vehicle.

12. The method of item 11, wherein the single unit dosage form further comprises an FLT3−ITD inhibitor, or wherein the HCK inhibitor is a dual HCK/FLT3−ITD inhibitor.

13. The method of any preceding item, wherein the HCK inhibitor is selected from RK-20449, RK-20693, RK-24466, RK-20444, RK-20445, and RK-20466.

14. The method of any one of items 3, 4, or 7-12, wherein the FLT3−ITD inhibitor is selected from AC220, sorafenib, PKC412, CEP-701, UNC2025, MLN518, KW-2449, AMG-925, sunitinib, SU5614, AC2206, crenolanib, and PLX3397.

15. The method of any preceding item, wherein the BCL-2 inhibitor is selected from AT-101, TW-37, TM-1206, gossypolic acid, gossypolonic acid, apogossypol, apogossypolone, A385358, ABT-737, ABT-263, ABT-199, WEHI-539, BXI-61, BXI-72, obatoclax, JY-1-106, and SAHB peptides.

16. The method of item 15, wherein the BCL-2 inhibitor is selected from gossypol, obatoclax, ABT-737, ABT-199, and ABT-263.

17. The method of item 16, wherein the BCL-2 inhibitor is ABT-199.

18. The method of item 17, wherein the HCK inhibitor is RK-20449 and the BCL-2 inhibitor is ABT-199.

19. The method of item 17, wherein the HCK inhibitor is RK-20693 and the BCL-2 inhibitor is ABT-199.

20. The method of item 17, wherein the FLT3−ITD inhibitor is AC220 and the BCL-2 inhibitor is ABT-199.

21. The method of item 16, wherein the FLT3−ITD inhibitor is SU5614 and the BCL-2 inhibitor is ABT-737.

22. The method of any preceding item, wherein the HCK inhibitor, and/or FLT3−ITD inhibitor, and/or the BCL-2 inhibitor is each present as a pharmaceutically acceptable salt.

23. The method of any preceding item, wherein the HCK inhibitor, and/or FLT3−ITD inhibitor, and/or the BCL-2 inhibitor is each present in a pharmaceutically acceptable composition.

Despite substantial clonal diversity defined by various combinations of somatic mutations, multi-kinase inhibition of SFKs and FLT3−ITD effectively reduced AML in vivo, and the addition of BCL2 inhibition synergistically eliminated resistant AML. In certain embodiments, the combined inhibition of anti-apoptotic and kinase signaling pathways through co-administration of RK-20449, a multi-kinase inhibitor of FLT3−ITD and HCK, and ABT-199, a small molecule inhibitor of BCL-2, led to successful elimination of human FLT3−ITD+AML in vivo despite substantial patient-to-patient heterogeneity and clonal diversity within individual patients.

### BRIEF DESCRIPTION OF DRAWINGS

FIG. 1 depicts mutational profiling of patient-derived subpopulations sorted by surface phenotype; the subpopulation undergoing NSG xenotransplantation to determine in vivo cell fate; and single-cell mutational profiling that was performed for patient-derived subpopulations with known cell fates and recipient-derived human multilineage hematopoietic cells and AML cells. FIG. 1A depicts mutational profiling of patient-derived subpopulations sorted by surface phenotype. FIG. 1B depicts the sub-population undergoing NSG xenotransplantation to determine in vivo cell fate. If repopulation by multilineage hematopoiesis occurred, the transplanted subpopulation contained hematopoietic stem cells (HSCs); if xenotransplantation resulted in AML engraftment, the transplanted subpopulation contained leukemia-initiating cells (LICs). FIG. 1C depicts single-cell mutational profiling that was performed for patient-derived subpopulations with known cell fates and recipient-derived human multilineage hematopoietic cells and AML cells.

FIG. 2-1 depicts identification of hematopoietic subpopulations in two patients (patients 21 and 20).

FIG. 2-2 depicts identification of hematopoietic subpopulations in two patients (patients 13 and 1). FIGS. 2A-2D depict identification of hematopoietic subpopulations in four patients. In each patient sample, T and B lymphoid populations and non-T non-B cells with distinct CD34 and CD38 surface expression were identified. Heat maps represent variant allele frequencies of NPM1, DNMT3A, CEBPA, IDH1, IDH2, TET2 and WT1 genes in indicated subpopulations isolated from each patient. FLT3−ITD mutation was detected by PCR. FIG. 2A is patient 21; FIG. 2B is patient 20; FIG. 2C is patient 13; and FIG. 2D is patient 1.

FIG. 3-1 depicts diverse subclones defined by various combinations of somatic mutations that were present in a patient-derived leukemia-initiating population and a multilineage-engrafting population, and engrafted AML and multilineage human hematopoietic cells.

FIG. 3-2 depicts diverse subclones defined by various combinations of somatic mutations that were present in a patient-derived leukemia-initiating population and a multilineage-engrafting population, and engrafted AML and multilineage human hematopoietic cells.

FIG. 3-3 depicts diverse subclones defined by various combinations of somatic mutations that were present in a patient-derived leukemia-initiating population and a multilineage-engrafting population, and engrafted AML and multilineage human hematopoietic cells.The leukemia-initiating population and the multilineage engrafting population from four patients, isolated by surface phenotype and functionally defined by xenotransplantation, underwent single cell sequencing for indicated genes and single cell PCR for FLT3−ITD. Each single cell is represented as a column of rectangles. The presence or absence of mutations in each indicated gene is shown by colors of the rectangles. FIG. 3A is patient 1; FIG. 3B is patient 21; FIG. 3C and 3D is patient 13; and FIG. 3E is patient 20.

FIG. 4 depicts variant allele frequencies of nine genes in patient- and recipient-derived AML cells in twelve AML cases represented as heat maps. “1” refers to patient-derived AML cells; “1” and “2”, primary and secondary recipient-derived AML cells, respectively. Blank squares indicate that the sequence could not be read. Sequencing for FLT3 was performed to identify non−ITD FLT3 mutations. All patient-derived and recipient-derived leukemia populations were positive for FLT3−ITD by PCR.

FIG. 5 depicts in vivo kinase inhibition that induces apoptosis of FLT3−ITD+AML cells with various combinations of co-existing somatic mutations and enhances dependence on Bcl-2 for survival. Each patient case was classified by in vivo RK-20449 treatment responses of recipients as follows: Complete responder (FIG. 5A), if all recipients treated showed residual BM human CD45+chimerism <5%; good responder (FIG. 5B), if the case did not meet the criterion for complete responder but all recipients showed <50% residual BM human CD45+; partial responder (FIG. 5C), if at least one of the recipients showed greater than 50% residual BM human CD45+. PB time-course of human AML chimerism (leftmost panels) for RK-20449 treated recipients and BM (middle panels) and spleen (rightmost panels) human AML chimerism at the time of sacrifice of RK-20449 treated and untreated recipients are shown. Pre-treatment PB human AML cell chimerism is shown at week 0. The numbers of recipients for each patient/each treatment group and pre-/post-treatment AML chimerisms are shown in FIG. 8.

FIG. 6A depicts the time courses of PB responses to in vivo treatments for four treatment groups (no treatment, ABT-199 alone, RK-20449 alone and RK-20449 and ABT-199). Recipients were phlebotomized weekly with time 0 representing pretreatment PB human CD45+AML chimerism. Final BM and spleen human AML chimerisms for nine cases that showed complete responses to combined treatment with RK-20449 and ABT-199.

FIG. 6B depicts human AML cell chimerisms in BM and spleen following in vivo treatment (no treatment, ABT-199 alone, RK-20449 alone, combination) for AML cases that showed complete responses. Each circle represents an AML-engrafted recipient.

FIG. 6C depicts that by serial transplantation, residual human AML-initiation capacity in human CD45+cells following in vivo treatment was assessed for four treatment groups. The transplanted AML cells were isolated from 2.5% of viable residual human AML cells remaining in recipients of each treatment group into each secondary recipient. Mean and SEM for human CD45+AML cell chimerism in the BM of secondary recipients are shown. Each circle represents a secondary recipient.

FIG. 6D depicts a schematic representation of the relationship between FLT3−ITD and other AML-associated mutations. A pre-leukemic cell population consists of HSCs carrying mutations in genes such as DNMT3A, TET2 and CEBPA in various combinations. These permission mutations may cooperate with FLT3−ITD for malignant transformation but do not preclude multilineage maturation to lymphoid and myeloid cells. On the other hand, FLT3−ITD possesses the greatest malignant potential in FLT3−ITD+AML patients. Acquisition of FLT3−ITD at the level of HSCs results in loss of multilineage differentiation capacity, converting pre-leukemic HSCs into LSCs. More mature progenitors and differentiated myeloid cells may also acquire FLT3−ITD and become LICs.

FIG. 7 provides clinical characteristics of patients. M0, M1, M2, M4, M5, M5a that indicated French-American-British classification at diagnosis. MDS, myelodysplastic syndrome; AML/MRC, acute myeloid leukemia with myelodysplasia-related changes; MF, myelofibrosis; CMML, chronic myelomonocytic leukemia; HSCT, hematopoietic stem cell transplantation; PB, peripheral blood; BM, bone marrow; IDA, idarubicin; AraC, cytarabine; uCBT, umbilical cord blood transplantation; CR, complete response; MIT, mitoxanthrone; GVHD, graft-versus-host disease; PBSCT, mobilized peripheral blood stem cell transplantation; AZA, azacytidine; HiDAC, high-dose cytarabine; CNS, central nervous system; GO, gemtuzumab ozogamicin; MUD, matched unrelated donor; BMT bone marrow transplantation; MRD, matched related donor.

FIG. 8 provides human AML chimerisms in PB, BM and spleen of AML-engrafted recipients treated with RK-20449 alone. The percent human CD45+cells in pre- and post-treatment PB and post-treatment BM and spleen are shown for recipients engrafted with human AML. PB, peripheral blood; BM, bone marrow; n, number of recipients; SEM, standard error of the mean; na, data not available. Response groups: Complete, post-treatment BM human CD45+<5% in every recipient treated; Good, post-treatment BM human CD45+<5% in all but one recipient treated; Partial, post-treatment BM human CD45+ is statistically significantly reduced compared with untreated group but >50% in one recipient treated.

FIG. 9 provides human AML chimerisms in PB, BM and spleen of AML-engrafted recipients treated with ABT-199 alone, RK-20449 alone or in combination. The percent human CD45+cells in pre- and post-treatment PB and post-treatment BM and spleen are shown for recipients engrafted with human AML. Untreated and RK-20449-treated recipient data are duplicated from FIG. 8 as comparison. Response groups: Complete, post-treatment BM human CD45+<5% in every recipient treated; Good, post-treatment BM human CD45+<5% in all but one recipient treated.

FIG. 10 provides statistics comparing pre- and post-treatment PB human AML chimerisms in recipients treated with ABT-199 alone, RK-20449 alone or in combination. The mean+/−s.e.m. of percent human CD45+cells in peripheral blood of recipients engrafted with human AML is shown. BM, number of recipients; p, paired two-tailed t-test comparing pre- and post-treatment PB human % CD45+; na, not powered to detect statistically significant differences.

FIG. 11 depicts statistics comparing BM and spleen human AML chimerisms in recipients treated with ABT-199 alone, RK-20449 alone or in combination. The mean+/−s.e.m. of percent human CD45+cells in recipients engrafted with human AML is shown. BM, bone marrow; n, number of recipients; p, unpaired two-tailed t-test between indicated parings; na, not powered to detect statistically significant differences.

### DESCRIPTION OF EMBODIMENTS

### Definitions

Unless defined otherwise, all technical and scientific terms used herein have the meaning commonly understood by a person skilled in the art of the present disclosure. The following references provide one of skill with a general definition of many of the terms used in this disclosure: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

In this disclosure, “comprises,” “comprising,” “containing” and “having” and the like can have the meaning ascribed to them in U.S. Patent law and can mean “ includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

As used herein, the term “detect” refers to identifying the presence, absence or amount of the analyte to be detected. The terms “eliminate” or “elimination” refer to an absence of analyte being detected. One of ordinary skill in the art readily appreciates that measurement methods inherently possess a limit(s) to its lowest and highest levels of detection. Thus, an indication of not detected as used herein is not to be construed to mean the analyte is not present at all. It is simply not present between the upper or lower limits of the detection method.

By “effective amount” or “therapeutically effective amount” is meant the amount of an active agent required to ameliorate the symptoms of a disease relative to an untreated subject. The effective amount of active agent(s) disclosed herein for therapeutic treatment of a disease varies depending upon a number of factors, including, but not limited to, the manner of administration, the age, body weight, and general health of the subject. The attending physician or veterinarian can decide the appropriate amount and dosage regimen.

The term “subject,” “patient” or “individual” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or other primates (e.g., cynomolgus monkeys, rhesus monkeys); mammals, including commercially relevant mammals such as cattle, pigs, horses, sheep, goats, cats, and/or dogs; and/or birds, including commercially relevant birds such as chickens, ducks, geese, quail, and/or turkeys.

As used herein, a treatment that “prevents” a disorder or condition refers to a treatment that, in a statistical sample, reduces the occurrence or frequency of the disorder or condition in the treated sample relative to an untreated control sample, or delays the onset or reduces the severity of one or more symptoms of the disorder or condition relative to the untreated control sample. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount. Prevention of an infection includes, for example, reducing the number of diagnoses of the infection in a treated population versus an untreated control population, and/or delaying the onset of symptoms of the infection in a treated population versus an untreated control population. Prevention of pain includes, for example, reducing the magnitude of, or alternatively delaying, pain sensations experienced by subjects in a treated population versus an untreated control population.

The term “treating” includes prophylactic and/or therapeutic treatments. The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic (i.e., it protects the host against developing the unwanted condition), whereas if it is administered after manifestation of the unwanted condition, the treatment is therapeutic (i.e., it is intended to diminish, ameliorate, or stabilize the existing unwanted condition or side effects thereof).

As used herein, the phrase “conjoint administration” refers to any form of administration of two or more different therapeutic compounds such that the second compound is administered while the previously administered therapeutic compound is still effective in the body (e.g., the two compounds are simultaneously effective in the patient, which may include synergistic effects of the two compounds). For example, the different therapeutic compounds can be administered either in the same formulation or in a separate formulation, either concomitantly or sequentially. In certain embodiments, the different therapeutic compounds can be administered within one hour, 12 hours, 24 hours, 36 hours, 48 hours, 72 hours, or a week of one another. Thus, an individual who receives such treatment can benefit from a combined effect of different therapeutic compounds.

### Overview

Despite substantial clonal diversity defined by various combinations of somatic mutations, multi-kinase inhibition of SFKs and FLT3−ITD effectively reduced AML in vivo, and the addition of BCL2 inhibition synergistically eliminated resistant AML. In certain embodiments, the combined inhibition of anti-apoptotic and kinase signaling pathways through co-administration of RK-20449, a multi-kinase inhibitor of FLT3−ITD and HCK, and ABT-199, a small molecule inhibitor of BCL-2, led to successful elimination of human FLT3−ITD+AML in vivo despite substantial patient-to-patient heterogeneity and clonal diversity within individual patients.

By integrating single cell genomics with in vivo functional assessment, patient-specific mutational profiles were identified that defined clonal architectures in preleukemic stem cells and leukemia-initiating cells in FLT3−ITD+AML. Among the mutations, FLT3−ITD possessed the greatest malignant potential in vivo, demarcating the transition from pre-leukemia to leukemia. Despite diverse co-existing mutations, RK-20449 effectively decreased the leukemic cell population in a PDX-model to below the limit of detection such as less than 0.01% remaining AML cells using flow cytometry and immunohistochemistry using multiple monoclonal antibodies. RK-20449 also enhanced BCL-2 dependence for leukemia cell survival, and co-inhibition of BCL-2 decreased the human leukemia cell count in vivo to below the limit of detection. Co-inhibition of these two pathways for malignant transformation and tumor survival is successful despite complex patient-specific mutational landscapes.

Further, specific inhibition of HCK can increase the potency of the combination of the FLT3−ITD inhibitors and BCL2 inhibitors described herein. HCK is a member of the Src-family of non-receptor tyrosine kinases, which plays many roles in signaling pathways involved in the regulation of cell processes. HCK is expressed in cells of hematopoietic origin, specifically myelomonocytic cells and B lymphocytes. It participates in phagocytosis, adhesion, migration, regulation of protrusion formation on cell membrane, lysosome exocytosis, podosome formation and actin polymerization. High levels of HCK are present in chronic myeloid leukemia and other hematologic tumors. HCK could also play a role in the genesis of acute myeloid leukemia.

In certain embodiments, provided herein is a method of co-inhibiting HCK and BCL-2 in a cell, comprising contacting the cell with an HCK inhibitor and a BCL-2 inhibitor, or a pharmaceutically acceptable composition thereof. In certain preferred embodiments, the method further comprises contacting the cell with a FLT3−ITD inhibitor, such as a dual HCK/FLT3−ITD inhibitor. In certain embodiments the cell is in vitro. In other embodiments, the cell is in vivo. In other embodiments, the cell is an acute myeloid leukemic cell.

In certain embodiments, provided herein is a method of killing a cell having a FLT3−ITD mutation, comprising contacting the cell with an HCK inhibitor and a BCL-2 inhibitor, or a pharmaceutically acceptable composition thereof. In certain preferred embodiments, the method further comprises contacting the cell with a FLT3−ITD inhibitor, such as a dual HCK/FLT3−ITD inhibitor. In certain embodiments the cell is in vitro. In other embodiments, the cell is in vivo. In other embodiments, the cell is an acute myeloid leukemic cell.

In certain embodiments, provided herein is a method of treating acute myeloid leukemia, comprising conjointly administering to a subject an HCK inhibitor and a BCL-2 inhibitor, or a pharmaceutically acceptable composition thereof. In certain preferred embodiments, the method further comprises conjointly administering to a subject a FLT3−ITD inhibitor. In other preferred embodiments, the HCK inhibitor is a dual HCK/FLT3−ITD inhibitor. Other embodiments provide a composition comprising a therapeutically effective amount of an HCK inhibitor and a BCL-2 inhibitor, or a pharmaceutically acceptable composition thereof, for the treatment of acute myeloid leukemia. In certain preferred embodiments, the composition further comprises a therapeutically effective amount of a FLT3−ITD inhibitor. In other preferred embodiments, the HCK inhibitor is a dual HCK/FLT3−ITD inhibitor. In other embodiments, provided herein is the use of a therapeutically effective amount of an HCK inhibitor and a BCL-2 inhibitor, or a pharmaceutically acceptable composition thereof, in the manufacture of a medicament for the treatment of acute myeloid leukemia. In certain preferred embodiments, the use further comprises a therapeutically effective amount of a FLT3−ITD inhibitor, while in other preferred embodiments, the HCK inhibitor is a dual HCK/FLT3−ITD inhibitor. In related embodiments, the invention provides the use of a therapeutically effective amount of an HCK inhibitor or a BCL-2 inhibitor in the manufacture of a medicament for use in a method of treating acute myeloid leukemia by the conjoint administration of an HCK inhibitor and a BCL-2 inhibitor. In certain preferred embodiments, the method further comprises conjointly administering a therapeutically effective amount of a FLT3−ITD inhibitor, while in other preferred embodiments, the HCK inhibitor is a dual HCK/FLT3−ITD inhibitor.

In certain embodiments, provided herein is a method of co-inhibiting HCK, FLT3−ITD, and BCL-2 in a cell, comprising contacting the cell with an HCK inhibitor, a FLT3−ITD inhibitor and a BCL-2 inhibitor, or a pharmaceutically acceptable composition thereof. In some embodiments, the HCK inhibitor and the FLT3−ITD inhibitor are provided as a dual HCK/FLT3−ITD inhibitor. In certain embodiments the cell is in vitro. In other embodiments, the cell is in vivo. In certain such embodiments, the cell is an acute myeloid leukemic cell.

In certain embodiments, provided herein is a method of killing a cell having an FLT3−ITD mutation, comprising contacting the cell with an HCK inhibitor, a FLT3−ITD inhibitor, and a BCL-2 inhibitor, or a pharmaceutically acceptable composition thereof. In some embodiments, the HCK inhibitor and the FLT3−ITD inhibitor are provided as a dual HCK/FLT3−ITD inhibitor. In certain embodiments the cell is in vitro. In other embodiments, the cell is in vivo. In certain such embodiments, the cell is an acute myeloid leukemic cell.

In certain embodiments, provided herein is a method of treating acute myeloid leukemia, comprising conjointly administering to a subject an HCK inhibitor, a FLT3−ITD inhibitor and a BCL-2 inhibitor, or a pharmaceutically acceptable composition thereof. In some embodiments, the HCK inhibitor and the FLT3−ITD inhibitor are provided as a dual HCK/FLT3−ITD inhibitor. Other embodiments provide composition comprising a therapeutically effective amount of an HCK inhibitor, a FLT3−ITD inhibitor and a BCL-2 inhibitor, or a pharmaceutically acceptable composition thereof, for the treatment of acute myeloid leukemia. In some embodiments, the HCK inhibitor and the FLT3−ITD inhibitor are provided as a dual HCK/FLT3−ITD inhibitor. In other embodiments, provided herein is the use of a therapeutically effective amount of an HCK inhibitor, a FLT3−ITD inhibitor and a BCL-2 inhibitor, or a pharmaceutically acceptable composition thereof, in the manufacture of a medicament for the treatment of acute myeloid leukemia. In some embodiments, the HCK inhibitor and the FLT3−ITD inhibitor are provided as a dual HCK/FLT3−ITD inhibitor. In related embodiments, the invention relates to the use of an HCK inhibitor, a FLT3−ITD inhibitor or a BCL-2 inhibitor in the manufacture of a medicament for treating acute myeloid leukemia in a method comprising the conjoint administration of the HCK inhibitor, the FLT3−ITD inhibitor or the BCL-2 inhibitor with the other of the HCK inhibitor, FLT3−ITD inhibitor and the BCL-2 inhibitor.

In certain embodiments, the HCK inhibitor is selected from RK-20449, RK-20693, RK-24466, RK-20444, RK-20445, and RK-20466. In other embodiments, the HCK inhibitor is selected from RK-20449, RK-20693, RK-24466, RK-20444, RK-20445, RK-20466, RK-20730, RK-20690, RK-20781, RK-20786, RK-20888, RK-20658, RK-20686, RK-20696, RK-20709, RK-20721, RK-20694, RK-20703, RK-20718, RK-20744, and compounds having HCK inhibitory activity disclosed in WO2014/017659.

In some embodiments, the FLT3−ITD inhibitor is selected from AC220, sorafenib, PKC412, CEP-701, UNC2025, MLN518, KW-2449 and AMG-925, sunitinib, SU5614, AC2206, crenolanib, and PLX3397. In certain preferred embodiments, the HCK inhibitor is RK-20449. In other embodiments, the FLT3−ITD inhibitor is AC220.

In other embodiments, the BCL-2 inhibitor is selected from AT-101, TW-37, TM-1206, gossypol, gossypolic acid, gossypolonic acid, apogossypol, apogossypolone, A385358, ABT-737, ABT-263, ABT-199, WEHI-539, BXI-61, BXI-72, obatoclax, ONT-701, S1, JY-1-106, and SAHB (stabilized α\och-helix of Bcl-2 domains) peptides. In certain embodiments, the BCL-2 inhibitor is selected from gossypol, obatoclax, ABT-737, ABT-199, and ABT-263. In certain preferred embodiments, the BCL-2 inhibitor is ABT-199.

Other BCL-2 inhibitors contemplated herein include, but are not limited to, those disclosed in Anderson, et al. Seminars in Hematology 2014 51:219-227; Bajwa et al. Expert Opin Ther. Pat. 2012 22:37-55; Oltersdorf, T., et al. Nature 2005 435:677-681; Tse, C. et al. Cancer Research 2008 68:3421-3428; Souers, A. J. et al. Nature Medicine 2013 19:202-208; Nguyen, M. et al. PNAS 2007 104:19512-19517; Zhang, A. et al. Int'l J. Cancer 2011 128:1724-1735; and Wei, J. et al. J. Med. Chem. 2009 52:4511-4523.

In certain preferred embodiments, the HCK inhibitor is RK-20449 and the BCL-2 inhibitor is ABT-199. In other embodiments, the HCK inhibitor is RK-20693 and the BCL-2 inhibitor is ABT-199. In other embodiments, the FLT3−ITD inhibitor is AC220 and the BCL-2 inhibitor is ABT-199. In other embodiments, the FLT3−ITD inhibitor is SU5614 and the BCL-2 inhibitor is ABT-737.

In certain embodiments, the effective concentration to inhibit HCK is greater than the effective concentration to inhibit BCL-2. In other embodiments, the effective concentration to inhibit HCK is less than the effective concentration to inhibit BCL-2. In other embodiments, the effective concentration to inhibit FLT3−ITD and the effective concentration to inhibit HCK are substantially similar.

In certain embodiments, the effective concentration to inhibit FLT3−ITD is greater than the effective concentration to inhibit BCL-2. In other embodiments, the effective concentration to inhibit FLT3−ITD is less than the effective concentration to inhibit BCL-2. In other embodiments, the effective concentration to inhibit FLT3−ITD and the effective concentration to inhibit BCL-2 are substantially similar.

In certain embodiments, the subject has malignant hematopoiesis and/or non-malignant multilineage hematopoiesis characterized by cells having one or more mutations in a gene selected from DNMT3A, IDH2, IDH1, NPM1, TET2, CEBPA, ASXL1, EZH2, SETBP1, SMC3, KIT, NRAS, and WT1.

### Combination Therapies

The invention generally relates to conjoint therapies involving a HCK inhibitor and a BCL-2 inhibitor, optionally with a FLT3−ITD inhibitor. In certain embodiments of the invention, the therapeutically effective amount of the FLT3−ITD and/or HCK inhibitor is less than its therapeutically effective amount would be where the BCL-2 inhibitor is not administered. Similarly, the therapeutically effective amount of the BCL-2 inhibitor may be less than its therapeutically effective amount would be where the FLT3−ITD inhibitor and/or HCK inhibitor is not administered, and the therapeutically effective amount of the HCK inhibitor may be less than its therapeutically effective amount would be where the FLT3−ITD inhibitor and/or BCL-2 inhibitor is not administered. In this way, undesired side effects associated with high doses of either agent may be minimized. Other potential advantages (including without limitation improved dosing regimens and/or reduced drug cost) will be apparent to those of skill in the art. The FLT3−ITD inhibitor, HCK inhibitor, and/or BCL-2 inhibitor may be administered separately from each other, e.g., as part of a multiple dose regimen. Alternatively, two or more inhibitors may be part of a single dosage form, e.g., mixed together in a single composition.

The amount of any of the FLT3−ITD, HCK, and BCL-2 inhibitors that can be combined with the carrier materials to produce a single dosage form will vary depending upon the host treated and the particular mode of administration. Preferably, compositions of this invention should be formulated so that a dosage of between about 0.01 - about 100 mg/kg body weight/day of an HCK inhibitor, a dosage of between about 0.01 - about 100 mg/kg body weight/day of a FLT3−ITD inhibitor, and a dosage of between about 0.01 - about 100 mg/kg body weight/day of a BCL-2 inhibitor can be administered. Also preferably, compositions of this invention should be formulated so that a dosage of between about 0.01 - about 100 mg/kg body weight/day of an HCK inhibitor and a dosage of between about 0.01 - about 100 mg/kg body weight/day of a BCL-2 inhibitor can be administered. In certain embodiments, a therapeutically effective amount of a dual HCK/FLT3 inhibitor or a BCL-2 inhibitor described herein may be administered alone or in combination with therapeutically effective amounts of other compounds useful for treating AML. In certain embodiments, a therapeutically effective amount of an HCK inhibitor, a FLT3−ITD inhibitor or a BCL-2 inhibitor described herein may be administered alone or in combination with therapeutically effective amounts of other compounds useful for treating AML.

### Compositions and Salts

In some embodiments, disclosed compounds can be in the form of a pharmaceutically acceptable composition. Disclosed herein are pharmaceutical compositions comprising a FLT3−ITD inhibitor, and/or HCK inhibitor, and/or a BCL-2 inhibitor as described herein and a pharmaceutically acceptable carrier.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, serum proteins such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-poly-oxypropylene-block polymers, polyethylene glycol and wool fat. Other pharmaceutically acceptable carriers, adjuvants or vehicles include water, saline and dimethyl-sulfoxide, as well as other hydrophobic or hydrophilic solvents.

Examples of suitable aqueous and nonaqueous carriers which can be employed in pharmaceutical compositions include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions can also contain adjuvants such as preservatives, wetting agents, emulsifying agents, dispersing agents, lubricants, and/or antioxidants. Prevention of the action of microorganisms upon the compounds described herein can be ensured by the inclusion of various antibacterial and antifungal agents, for example, paraben, chlorobutanol, phenol sorbic acid, and the like. It can also be desirable to include isotonic agents, such as sugars, sodium chloride, and the like into the compositions.

Methods of preparing these formulations or compositions include the step of bringing into association a compound described herein with the carrier and, optionally, one or more accessory ingredients. In general, the formulations are prepared by uniformly and intimately bringing into association a compound as disclosed herein with liquid carriers, or finely divided solid carriers, or both, and then, if necessary, shaping the product.

Preparations for such pharmaceutical compositions are well-known in the art. See, e.g., Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, New York, 1990; Katzung, ed., Basic and Clinical Pharmacology, Ninth Edition, McGraw Hill, 2003; Goodman and Gilman, eds., The Pharmacological Basis of Therapeutics, Tenth Edition, McGraw Hill, 2001; Remington's Pharmaceutical Sciences, 20th Ed., Lippincott Williams & Wilkins., 2000; Martindale, The Extra Pharmacopoeia, Thirty-Second Edition (The Pharmaceutical Press, London, 1999); all of which are incorporated by reference herein in their entirety. Except insofar as any conventional excipient medium is incompatible with the compounds provided herein, such as by producing any undesirable biological effect or otherwise interacting in a deleterious manner with any other component(s) of the pharmaceutically acceptable composition, the excipient's use is contemplated to be within the scope of this disclosure.

Provided herein are pharmaceutically acceptable salts which refer to those salts which are, within the scope of sound medical judgment, suitable for use in contact with the tissues of subjects without undue toxicity, irritation, allergic response and the like, and are commensurate with a reasonable benefit/risk ratio. Pharmaceutically acceptable salts are well known in the art. For example, Berge et al. describes pharmaceutically acceptable salts in detail in J. Pharmaceutical Sciences (1977) 66:1-19. Pharmaceutically acceptable salts of the compounds provided herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, besylate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. In some embodiments, organic acids from which salts can be derived include, for example, acetic acid, propionic acid, glycolic acid, pyruvic acid, oxalic acid, lactic acid, trifluoracetic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

The salts can be prepared in situ during the isolation and purification of the disclosed compounds, or separately, such as by reacting the free base or free acid of the compound with a suitable base or acid, respectively. Pharmaceutically acceptable salts derived from appropriate bases include alkali metal, alkaline earth metal, ammonium and N+(C₁₋₄alkyl)₄ salts. Representative alkali or alkaline earth metal salts include sodium, lithium, potassium, calcium, magnesium, iron, zinc, copper, manganese, aluminum, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate. Organic bases from which salts can be derived include, for example, primary, secondary, and tertiary amines, substituted amines, including naturally occurring substituted amines, cyclic amines, basic ion exchange resins, and the like, such as isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts.

### Administration

Compositions of the present invention may be administered orally, parenterally (including subcutaneous, intramuscular, intravenous and intradermal), by inhalation spray, topically, rectally, nasally, buccally, vaginally or via an implanted reservoir. In some embodiments, provided compounds or compositions are administrable intravenously and/or intraperitonally. In certain preferred embodiments, the disclosed methods include orally or parenterally administering any two of, or all three of, a FLT3−ITD inhibitor, an HCK inhibitor, and a BCL-2 inhibitor.

The term “parenteral,” as used herein, includes subcutaneous, intravenous, intramuscular, intraocular, intravitreal, intra-articular, intra-synovial, intrasternal, intrathecal, intrahepatic, intraperitoneal, intralesional and intracranial injection or infusion techniques. Preferably, the compositions are administered orally, subcutaneously, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

Pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added. In some embodiments, a provided oral formulation is formulated for immediate release or sustained/delayed release. In some embodiments, the composition is suitable for buccal or sublingual administration, including tablets, lozenges and pastilles. A compound disclosed herein can also be in micro-encapsulated form.

The amount of a compound of the present invention that may be combined with the carrier materials to produce a composition in a single dosage form will vary depending upon the subject being treated and the particular mode of administration. In certain embodiments, provided compositions should be formulated so that a dosage of between about 0.01 to about 100 mg/kg body weight/day of the compound can be administered to a subject receiving these compositions. In other embodiments, the dosage is from about 0.5 to about 100 mg/kg of body weight, or between about 1 mg and about 1000 mg/dose, about every 4 to 120 hours, or according to the requirements of the particular drug. Typically, the pharmaceutical compositions of this invention will be administered from about 1 to about 6 times per day.

In some embodiments, the compound is formulated for oral administration at a dosage of approximately 5 mg/kg to approximately 10 mg/kg, preferably at a dosage of approximately 7.5 mg/kg.

It should also be understood that a specific dosage and treatment regimen for any particular subject will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

Upon improvement of a subject's condition, a maintenance dose of a compound, composition or combination of this invention may be administered, if necessary. Subsequently, the dosage or frequency of administration, or both, may be reduced, as a function of the symptoms, to a level at which the improved condition is retained when the symptoms have been alleviated to the desired level. Subjects may, however, require intermittent treatment on a long-term basis upon any recurrence of disease symptoms.

### INCORPORATION BY REFERENCE

All publications, patents, and patent applications mentioned in this specification are herein incorporated by reference to the same extent as if each individual publication, patent, or patent application was specifically and individually indicated to be incorporated by reference. In case of conflict, the present application, including any definitions herein, will control.

### Examples

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions with art-recognized alternatives and using no more than routine experimentation, are within the scope of the present application.

The following examples are put forth so as to provide those of ordinary skill in the art with a complete disclosure and Description of how to make and use the methods of the invention, and are not intended to limit the scope of what the inventor(s) regard(s) as the invention.

Unless noted otherwise, the starting materials for the experiments described herein were obtained from commercial sources or known procedures and were used without further modification.

### General Methods

### Compounds

The following compounds can be synthesized according to, e.g., the methods disclosed in WO2014/017659.

RK-20449 (also known as A 419259):

7-((1R,4R)-4-(4-methylpiperazin-l-yl)cyclohexyl)-5-(4-phenoxyphenyl)-7H-pyrrolo[ 2,3-d]pyrimidin-4-amine

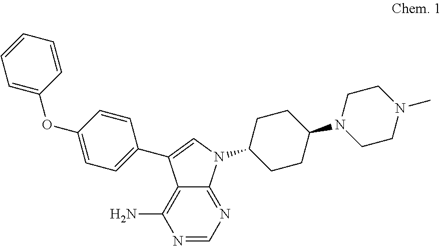 [SMILES: CN1CCN([C@H]2CC[C@H](n3cc(-c4ccc(Oc5ccccc5)cc4)c4c(N)ncnc43)CC2)CC1]

RK-20693:

N-(4-(4-amino-7-(trans-4-(4-methylpiperazin-1-yl)cyclohexyl)-7H-pyrrolo[2,3-d]pyrimidin-5-yl)phenyl)- 3-phenylpropanamide

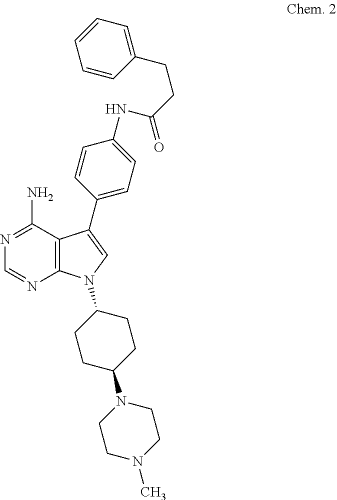 [SMILES: CN1CCN([C@H]2CC[C@H](n3cc(-c4ccc(NC(=O)CCc5ccccc5)cc4)c4c(N)ncnc43)CC2)CC1]

RK-24466:

7-c yclopentyl-5-(4-phenoxyphenyl)-7H-pyrrolo[2,3-d]pyrimidin-4-amine

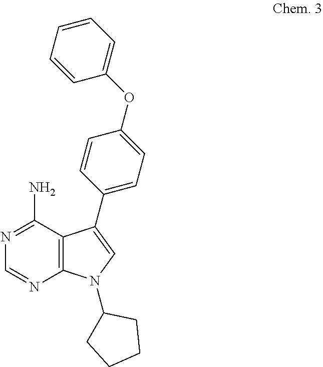 [SMILES: Nc1ncnc2c1c(-c1ccc(Oc3ccccc3)cc1)cn2C1CCCC1]

RK-20444:

1-(4-(4-amino-7-cyclopentyl-7H-pyrrolo[2,3-d]pyrimidin-5-yl)phenyl)-3-benzylurea

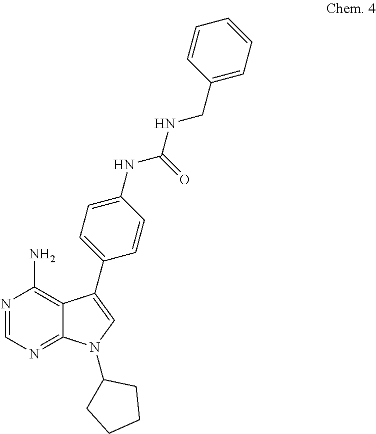 [SMILES: Nc1ncnc2c1c(-c1ccc(NC(=O)NCc3ccccc3)cc1)cn2C1CCCC1]

RK-20445:

N-(4-(4-amino-7-cyclopentyl-7H-pyrrolo[2,3-d]pyrimidin-5-yl)phenyl)benzamide

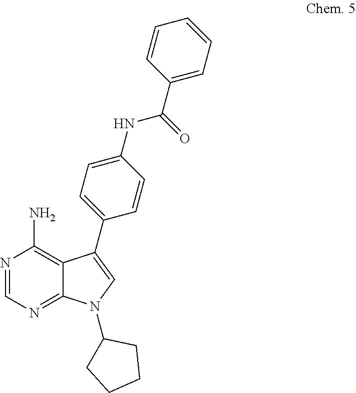 [SMILES: Nc1ncnc2c1c(-c1ccc(NC(=O)c3ccccc3)cc1)cn2C1CCCC1]

RK-20466:

7-cyclopentyl-5-(4-phenoxyphenyl)-7H-pyrrolo[2,3-d]pyrimidin-4-amine

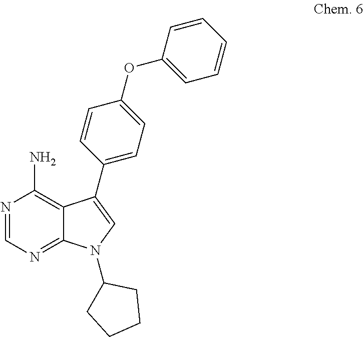 [SMILES: Nc1ncnc2c1c(-c1ccc(Oc3ccccc3)cc1)cn2C1CCCC1]

RK-20730:

N-(4-(4-amino-7-((1R,4R)-4-(3-methyl-5,6-dihydro-[1,2,4] triazolo [4,3-a]pyrazin-7(8H)-yl)cyclohexyl)-7H-pyrrolo [2,3-d]pyrimidin-5-yl)-2-methoxyphenyl)-1-methyl-1H-indole-2-carboxamide

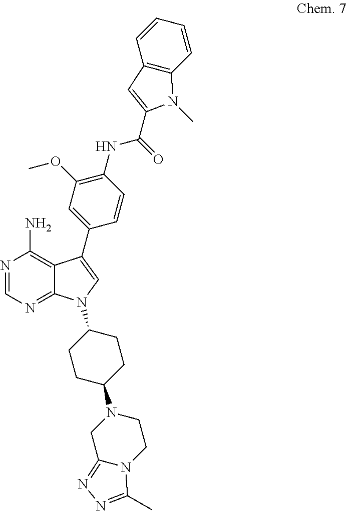 [SMILES: COc1cc(-c2cn([C@H]3CC[C@H](N4CCn5c(C)nnc5C4)CC3)c3ncnc(N)c23)ccc1NC(=O)c1cc2ccccc2n1C]

RK-20690:

7-41R,4R)-4-(8-methyl-3,8-diazabicyclo[3.2.1]octan-3-yl)cyclohexyl)-5-(4-phenoxyphenyl)-7H-pyrrolo[2,3-d]pyrimidin-4-amine

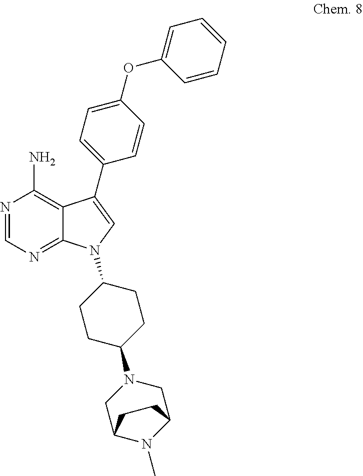 [SMILES: CN1[C@@H]2CC[C@H]1CN([C@H]1CC[C@H](n3cc(-c4ccc(Oc5ccccc5)cc4)c4c(N)ncnc43)CC1)C2]

RK-20781:

7-(1-(8-methyl-8-azabicyclo[3.2.1]octan-3-yl)piperidin-4-yl)-5-(4-phenoxyphenyl)-7H-pyrrolo[2,3-d]pyrimidin-4-amine

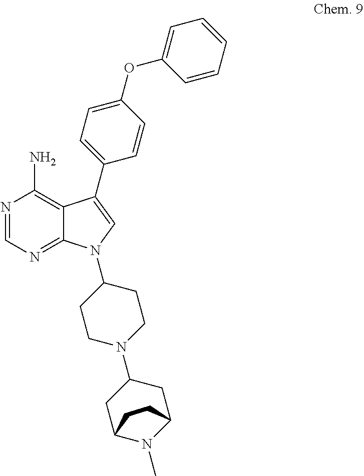

RK-20888:

1-((1S,4S)-4-((8-methyl-8-azabicyclo[3.2.1]octan-3-yl)amino)cyclohexyl)-3-(4-phenoxyphenyl)-1H-pyrazolo[3,4-d]pyrimidin-4-amine

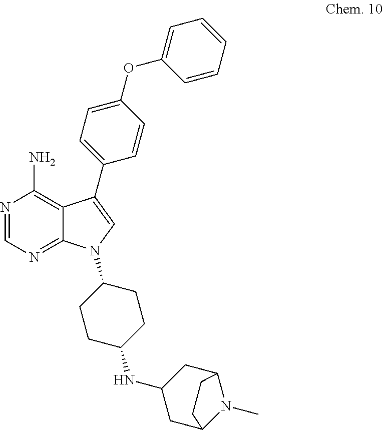 [SMILES: CN1C2CCC1CC(N[C@H]1CC[C@@H](n3cc(-c4ccc(Oc5ccccc5)cc4)c4c(N)ncnc43)CC1)C2]

RK-20658:

5-(4-phenoxyphenyl)-7-((1r,4r)-4-(piperazin-1-yl)cyclohexyl)-7H-pyrrolo[2,3-d]pyrimidin-4-amine

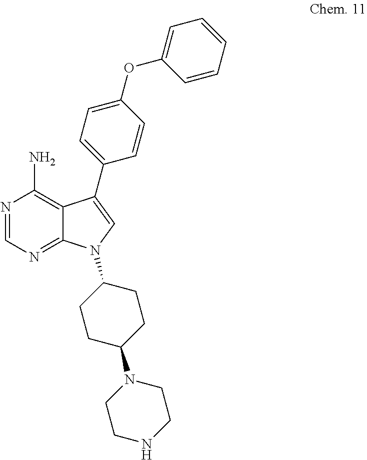 [SMILES: Nc1ncnc2c1c(-c1ccc(Oc3ccccc3)cc1)cn2[C@H]1CC[C@H](N2CCNCC2)CC1]

RK-20686:

7-((1R,4R)-4-(3-methyl-5,6-dihydro-[1,2,4]triazolo[4,3-a]pyrazin-7(8H)-yl)cyclohexyl)-5-(4-phenoxyphenyl)-7H-pyrrolo[2,3-d]pyrimidin-4-amine

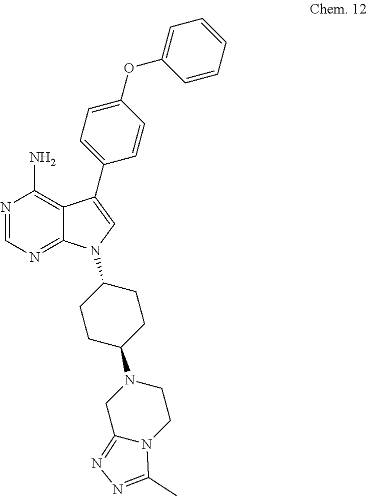 [SMILES: Cc1nnc2n1CCN([C@H]1CC[C@H](n3cc(-c4ccc(Oc5ccccc5)cc4)c4c(N)ncnc43)CC1)C2]

RK20696:

5-(4-phenoxyphenyl)-7-((1S,4S)-4-((pyridin-3-ylmethyl)amino)cyclohexyl)-7H-pyrrolo[2,3-d]pyrimidin-4-amine

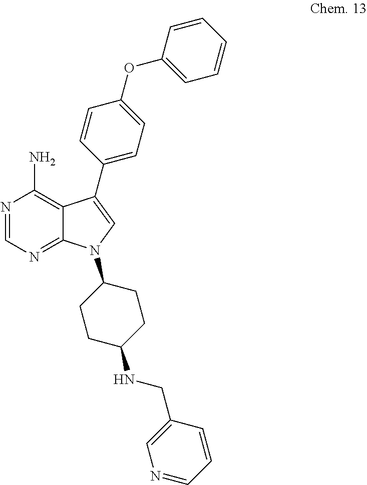 [SMILES: Nc1ncnc2c1c(-c1ccc(Oc3ccccc3)cc1)cn2[C@H]1CC[C@@H](NCc2cccnc2)CC1]

RK-20709:

7-([1,4′-bipiperidin]-4-yl)-5-(4-phenoxyphenyl)-7H-pyrrolo[2,3-d]pyrimidin-4-amine

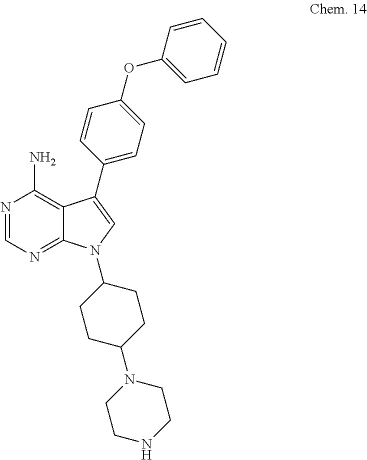 [SMILES: Nc1ncnc2c1c(-c1ccc(Oc3ccccc3)cc1)cn2C1CCC(N2CCNCC2)CC1]

RK-20721:

2-(((1R,4R)-4-(4-amino-5-(4-phenoxyphenyl)-7H-pyrrolo[2,3-d]pyrimidin-7-yl)cyclohexyl)amino)acetic acid trihydrochloride

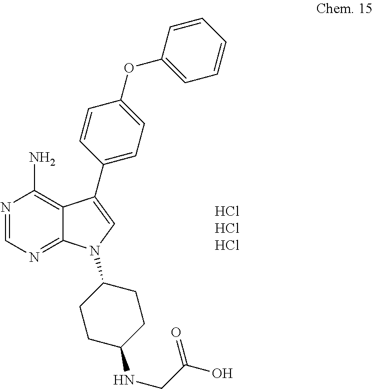 [SMILES: Nc1ncnc2c1c(-c1ccc(Oc3ccccc3)cc1)cn2[C@H]1CC[C@H](NCC(=O)O)CC1]

RK-20694:

5-(4-phenoxyphenyl)-7-((1S,4S-4-((pyridin-4-ylmethyl)amino)cyclohexyl)-7H-pyrrolo[2,3-d]pyrimidin-4-amine

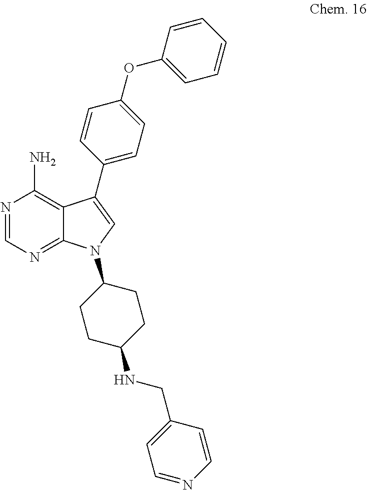 [SMILES: Nc1ncnc2c1c(-c1ccc(Oc3ccccc3)cc1)cn2[C@H]1CC[C@@H](NCc2ccncc2)CC1]

RK-20703:

5-(4-phenoxyphenyl)-7-((1S,4S)-4-((tetrahydro-2H-pyran-4-yl)amino)cyclohexyl)-7H-pyrrolo[2,3-d]pyrimidin-4-amine

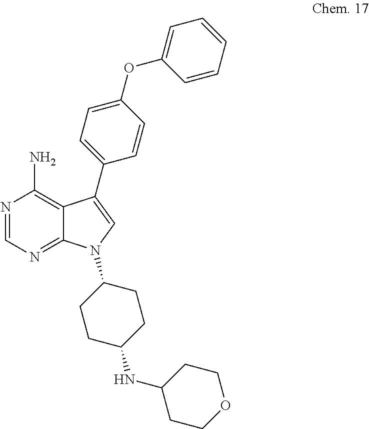 [SMILES: Nc1ncnc2c1c(-c1ccc(Oc3ccccc3)cc1)cn2[C@H]1CC[C@@H](NC2CCOCC2)CC1]

RK-20718:

2-(((1S,4S)-4-(4-amino-5-(4-phenoxyphenyl)-7H-pyrrolo[2,3-d]pyrimidin-7-yl)cyclohexyl)amino)acetic acid trihydrochloride

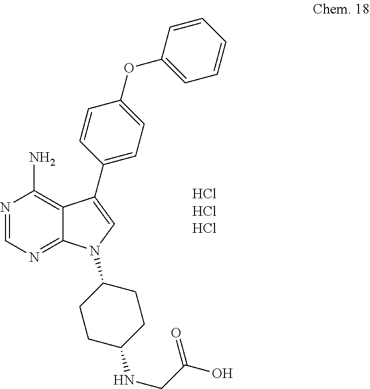 [SMILES: Nc1ncnc2c1c(-c1ccc(Oc3ccccc3)cc1)cn2[C@H]1CC[C@@H](NCC(=O)O)CC1]

RK-20719:

2-(((1S ,4S)-4-(4-amino-5-(4-phenoxyphenyl)-7H-pyrrolo [2,3-d]pyrimidin-7-yl)cyclohexyl)amino)acetamide

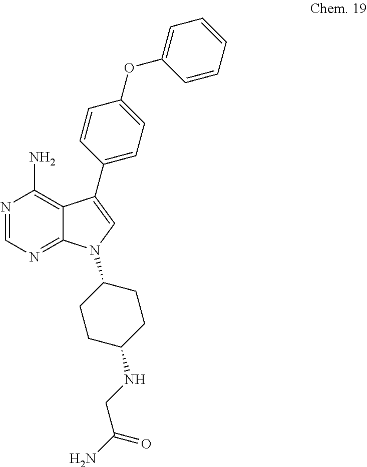 [SMILES: NC(=O)CN[C@H]1CC[C@@H](n2cc(-c3ccc(Oc4ccccc4)cc3)c3c(N)ncnc32)CC1]

RK-20722:

2-(((1R,4R)-4-(4-amino-5-(4-phenoxyphenyl)-7H-pyrrolo[2,3-d]pyrimidin-7-yl)cyclohexyl)amino)acetamide

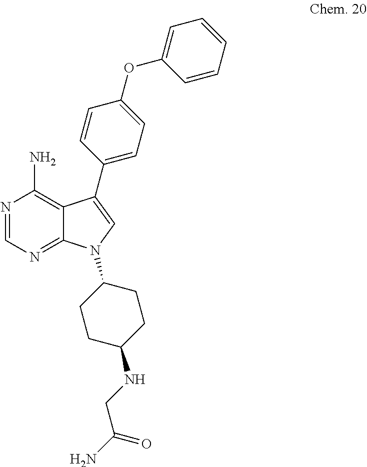 [SMILES: NC(=O)CN[C@H]1CC[C@H](n2cc(-c3ccc(Oc4ccccc4)cc3)c3c(N)ncnc32)CC1]

RK-20752:

N-((1S ,4S)-4-(4-amino-5-(4-phenoxyphenyl)-7H-pyrrolo[2,3-d]pyrimidin-7-yl)cyclohexyl)isonicotinamide

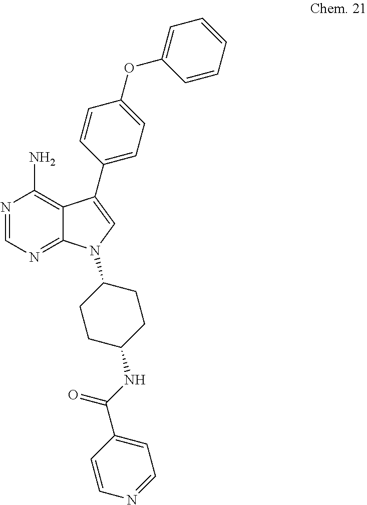 [SMILES: Nc1ncnc2c1c(-c1ccc(Oc3ccccc3)cc1)cn2[C@H]1CC[C@@H](NC(=O)c2ccncc2)CC1]

RK-20952:

7-((1R,4R)-4-(4-(tert-butyl)piperazin-1-yl)cyclohexyl)-5-(4-phenoxyphenyl)-7H-pyrrolo[2,3-d]pyrimidin-4-amine

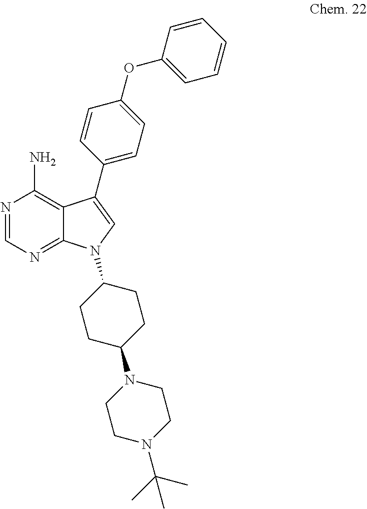 [SMILES: CC(C)(C)N1CCN([C@H]2CC[C@H](n3cc(-c4ccc(Oc5ccccc5)cc4)c4c(N)ncnc43)CC2)CC1]

RK-20618:

N-(4-(4-amino-1-((1R,4R)-4-(4-methylpiperazin-1-yl)cyclohexyl)-1H-pyrazolo[3,4-d]pyrimidin-3-yl)-2-methoxyphenyl)-1-methyl-1H-indole-2-carboxamide

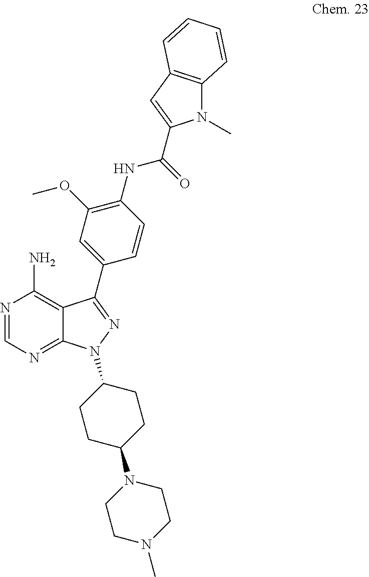 [SMILES: COc1cc(-c2nn([C@H]3CC[C@H](N4CCN(C)CC4)CC3)c3ncnc(N)c23)ccc1NC(=O)c1cc2ccccc2n1C]

RK-20725:

1-((1R,4R)-4-(8-methyl-3,8-diazabicyclo[3.2.1]octan-3-yl)cyclohexyl)-3-(4-phenoxyphenyl)-1H-pyrazolo[3,4-d]pyrimidin-4-amine

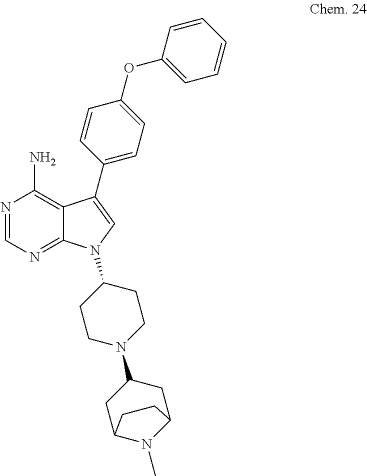

RK-20729:

N-(4-(4-amino-7((1S,4S)-4-(3-methyl-5,6-dihydro-[1,2,4]triazolo[4,3-a]pyrazin-7(8H)-yl)cyclohexyl)-7H-pyrrolo [2,3-d]pyrimidin-5-yl)-2-methoxyphenyl)-1-methyl-1H-indole-2-carboxamide

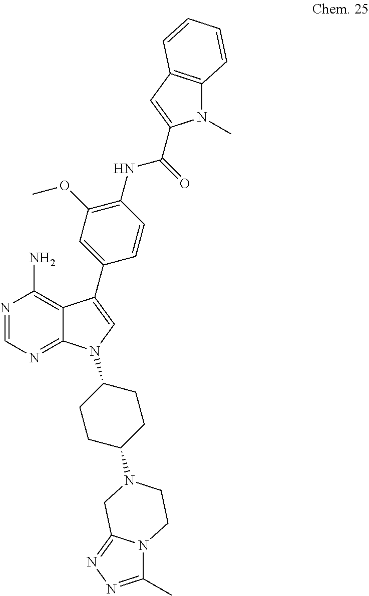 [SMILES: COc1cc(-c2cn([C@H]3CC[C@@H](N4CCn5c(C)nnc5C4)CC3)c3ncnc(N)c23)ccc1NC(=O)c1cc2ccccc2n1C]

RK-20732:

N-(4-(4-amino-7-((1r,4r)-4-(4-methylpiperazin-1-yl)cyclohexyl)-7H-pyrrolo[2,3-d]pyrimidin-5-yl)phenyl)-1-methyl-1H-indole-2-carboxamide

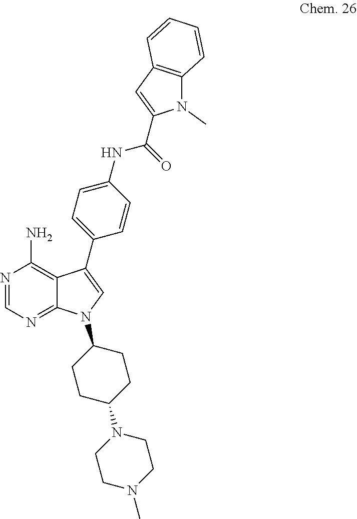 [SMILES: CN1CCN([C@H]2CC[C@H](n3cc(-c4ccc(NC(=O)c5cc6ccccc6n5C)cc4)c4c(N)ncnc43)CC2)CC1]

RK-20746:

N-((1R,4R)-4-(4-amino-5-(4-phenoxyphenyl)-7H-pyrrolo[2,3-d]pyrimidin-7-yl)cyclohexyl)picolinamide

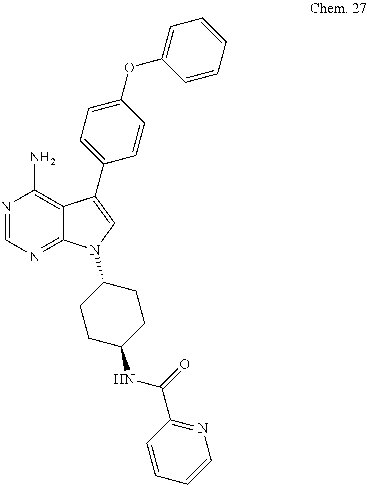 [SMILES: Nc1ncnc2c1c(-c1ccc(Oc3ccccc3)cc1)cn2[C@H]1CC[C@H](NC(=O)c2ccccn2)CC1]

RK-20755:

N-((1R,4R)-4-(4-amino-5-(4-phenoxyphenyl)-7H-pyrrolo[2,3-d]pyrimidin-7-yl)cyclohexyl)nicotinamide

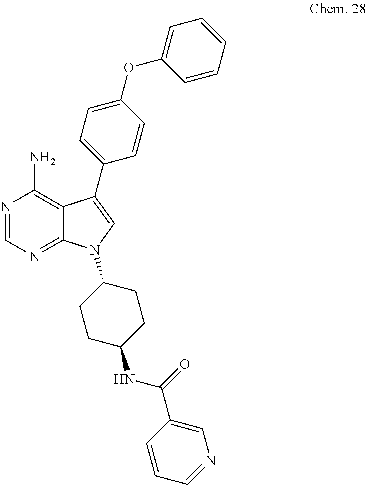 [SMILES: Nc1ncnc2c1c(-c1ccc(Oc3ccccc3)cc1)cn2[C@H]1CC[C@H](NC(=O)c2cccnc2)CC1]

RK-20768:

N-((1R,4R)-4-(4-amino-3-(4-phenoxyphenyl)-1H-pyrazolo[3,4-d]pyrimidin-1-yl)cyclohexyl)isonicotinamide

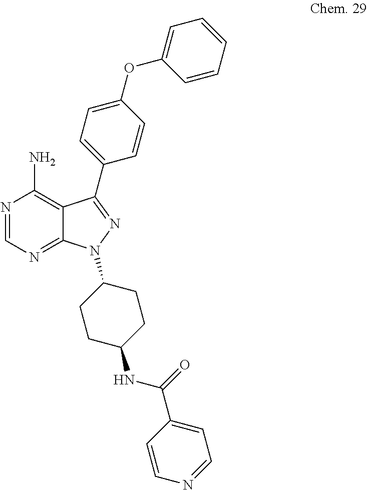 [SMILES: Nc1ncnc2c1c(-c1ccc(Oc3ccccc3)cc1)nn2[C@H]1CC[C@H](NC(=O)c2ccncc2)CC1]

RK20770:

N-(4-(4-amino-1-((1r, 4-(8-methyl-3,8-diazabicyclo[3.2.1]octan-3-yl)cyclohexyl)-1H-pyrazolo[3,4-d]pyrimidin-3-yl)phenyl)picolinamide

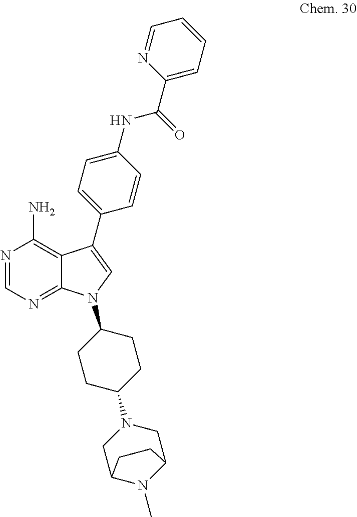 [SMILES: CN1C2CCC1CN([C@H]1CC[C@H](n3cc(-c4ccc(NC(=O)c5ccccn5)cc4)c4c(N)ncnc43)CC1)C2]

RK-20775:

N-((1R,4R)-4-(4-amino-3-(4-phenoxyphenyl)-1H-pyrazolo[3,4-d]pyrimidin-1-yl)cyclohexyl)nicotinamide

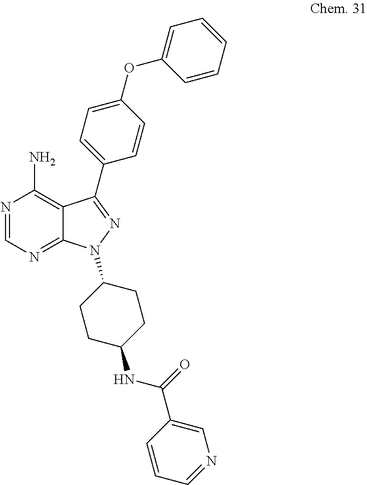 [SMILES: Nc1ncnc2c1c(-c1ccc(Oc3ccccc3)cc1)nn2[C@H]1CC[C@H](NC(=O)c2cccnc2)CC1]

RK-20777:

N-((1R,4R)-4-(4-amino-3-(4-phenoxyphenyl)-1H-pyrazolo[3,4-d]pyrimidin-1-yl)cyclohexyl)picolinamide

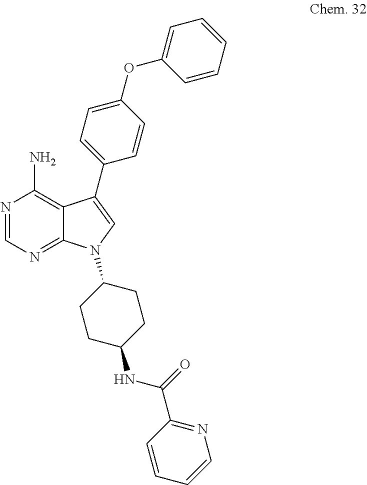 [SMILES: Nc1ncnc2c1c(-c1ccc(Oc3ccccc3)cc1)cn2[C@H]1CC[C@H](NC(=O)c2ccccn2)CC1]

RK-20791:

N-(4-(4-amino-1((1R,4R)-4-(8-methyl-3,8-diazabicyclo[3.2.1]octan-3-yl)cyclohexyl)-1H-pyrazolo[3,4-d]pyrimidin-3-yl)phenyl)-1-methyl-1H-indole-2-carboxamide

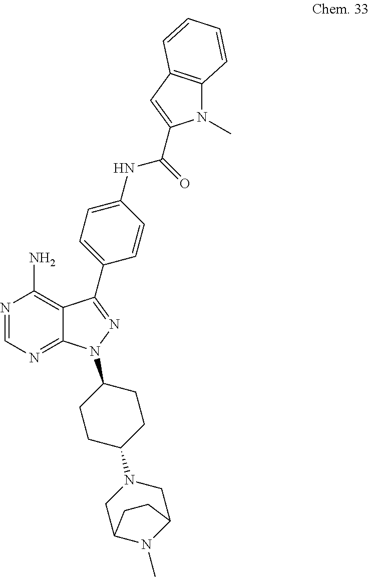 [SMILES: CN1C2CCC1CN([C@H]1CC[C@H](n3nc(-c4ccc(NC(=O)c5cc6ccccc6n5C)cc4)c4c(N)ncnc43)CC1)C2]

RK-20798:

N-(4-(4-amino-1-(1-(8-methyl-8-azabicyclo[3.2.1]octan-3-yl)piperidin-4-yl)-1H-pyrazolo[3,4-d]pyrimidin-3-yl)phenyl)-1-methyl-1H-indole-2-carboxamide

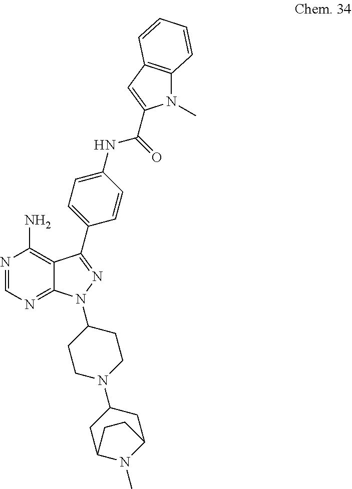 [SMILES: CN1C2CCC1CC(N1CCC(n3nc(-c4ccc(NC(=O)c5cc6ccccc6n5C)cc4)c4c(N)ncnc43)CC1)C2]

RK-20819:

N-(4-(4-amino-7-((1S,4S)-4-(4-methylpiperazin-1-yl)cyclohexyl)-7H-pyrrolo[2,3-d]pyrimidin-5-yl)-2-methoxyphenyl)-1-methyl-1H-indole-2-carboxamide

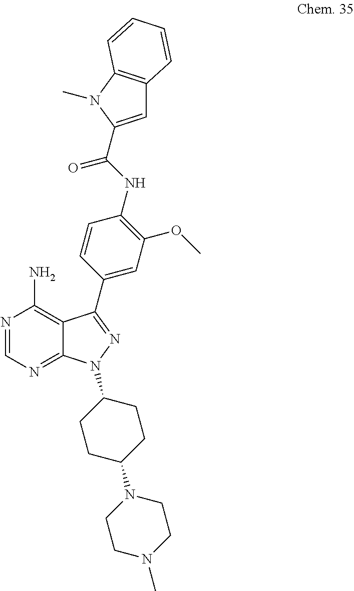 [SMILES: COc1cc(-c2nn([C@H]3CC[C@@H](N4CCN(C)CC4)CC3)c3ncnc(N)c23)ccc1NC(=O)c1cc2ccccc2n1C]

RK-20820:

N-(4-(4-amino-7-((1R,4R)-4-(4-methylpiperazin-1-yl)cyclohexyl)-7H-pyrrolo [2,3-d]pyrimidin-5-yl)-2-methoxyphenyl)-1-methyl-1H-indole-2-carboxamide

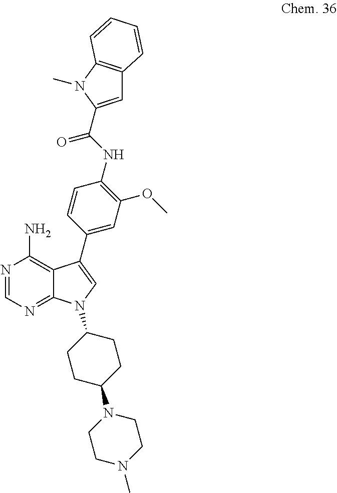 [SMILES: COc1cc(-c2cn([C@H]3CC[C@H](N4CCN(C)CC4)CC3)c3ncnc(N)c23)ccc1NC(=O)c1cc2ccccc2n1C]

RK-20824:

N-(4-(4-amino-1-((1R,4R)-4-(4-methylpiperazin-1-yl)cyclohexyl)-1H-pyrazolo[3,4-d]pyrimidin-3-yl)phenyl)-1-methyl-1H-indole-2-carboxamide

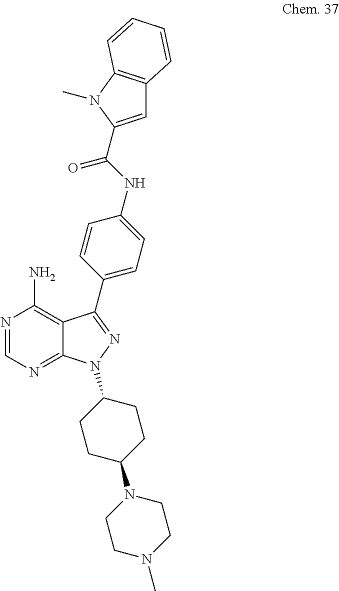 [SMILES: CN1CCN([C@H]2CC[C@H](n3nc(-c4ccc(NC(=O)c5cc6ccccc6n5C)cc4)c4c(N)ncnc43)CC2)CC1]

RK-20826:

N-(4-(4-amino-1-((1R,4R)-4-(3-methyl-5,6-dihydro-[1,2,4]triazolo[4,3-a]pyrazin-7(8H)-yl)cyclohexyl)-1H-pyrazolo[3,4-d]pyrimidin-3-yl)-2-methoxyphenyl)-1-methyl-1H-indole-2-carboxamide

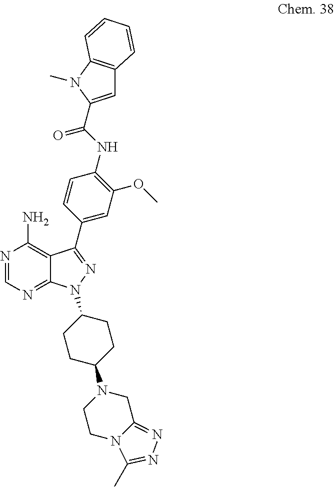 [SMILES: COc1cc(-c2nn([C@H]3CC[C@H](N4CCn5c(C)nnc5C4)CC3)c3ncnc(N)c23)ccc1NC(=O)c1cc2ccccc2n1C]

RK-20901:

N-(4-(4-amino-7-((1R,4R)-4-(3-(trifluoromethyl)-5,6-dihydro-[1,2,4]triazolo[4,3-a]pyrazin-7(8H)-yl)cyclohexyl)-7H-pyrrolo[2,3-d]pyrimidin-5-yl)-2-methoxyphenyl)-1-methyl-1H-indole-2-carboxamide

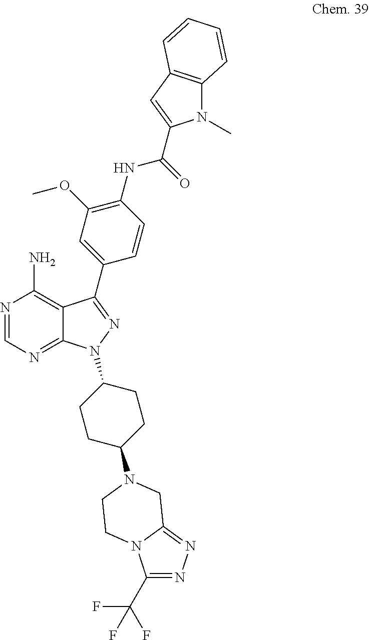 [SMILES: COc1cc(-c2nn([C@H]3CC[C@H](N4CCn5c(nnc5C(F)(F)F)C4)CC3)c3ncnc(N)c23)ccc1NC(=O)c1cc2ccccc2n1C]

RK-20908:

N-(4-(4-amino-7-((1s,4s)-4-(3-ethyl-5,6-dihydro-[1,2,4]triazolo[4,3-a]pyrazin-7(8H)-yl)cyclohexyl)-7H-pyrrolo[2,3-d]pyrimidin-5-yl)-2-methoxyphenyl)-1-methyl-1H-indole-2-carboxamide

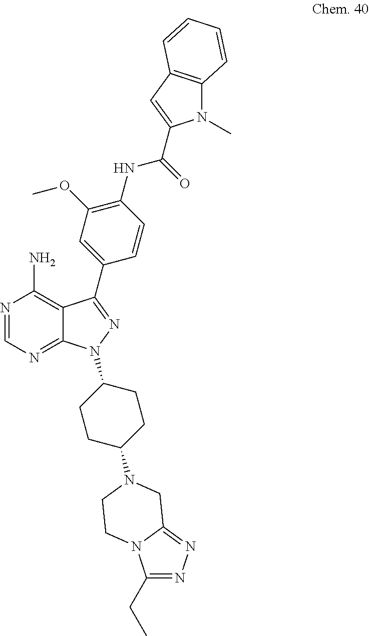 [SMILES: CCc1nnc2n1CCN([C@H]1CC[C@@H](n3nc(-c4ccc(NC(=O)c5cc6ccccc6n5C)c(OC)c4)c4c(N)ncnc43)CC1)C2]

RK-20909:

N-(4-(4-amino-7-((1r,4r)-4-(3-ethyl-5,6-dihydro- [1,2,4]triazolo [4,3-a]pyrazin-7(8H)-yl)cyclohexyl)-7H-pyrrolo [2,3-d]pyrimidin-5-yl)-2-methoxyphenyl)-1-methyl-1H-indole-2-carboxamide

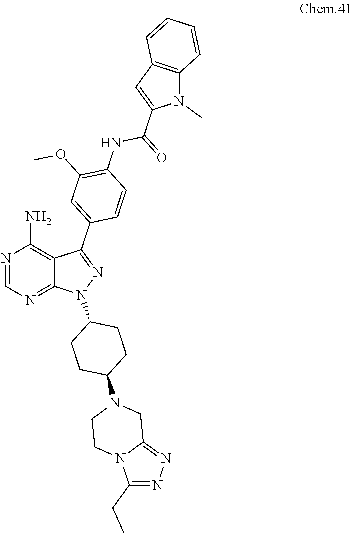 [SMILES: CCc1nnc2n1CCN([C@H]1CC[C@H](n3nc(-c4ccc(NC(=O)c5cc6ccccc6n5C)c(OC)c4)c4c(N)ncnc43)CC1)C2]

RK-20918:

N-(4-(4-amino-7-((1s,4s)-4-(3-isopropyl-5,6-dihydro- [1,2,4]triazolo [4,3-a]pyrazin-7(8H)-yl)cyclohexyl)-7H-pyrrolo [2,3-d]pyrimidin-5-yl)-2-methoxyphenyl)-1-methyl-1H-indole-2-carboxamide

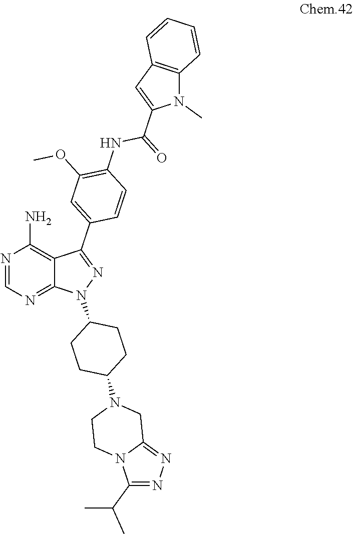 [SMILES: COc1cc(-c2nn([C@H]3CC[C@@H](N4CCn5c(nnc5C(C)C)C4)CC3)c3ncnc(N)c23)ccc1NC(=O)c1cc2ccccc2n1C]

RK-20919:

N-(4-(4-amino-7-((1R,4R)-4-(3-isopropyl-5,6-dihydro- [1,2,4]triazolo [4,3-a]pyrazin-7(8H)-yl)cyclohexyl)-7H-pyrrolo[2,3-d]pyrimidin-5-yl)-2-methoxyphenyl)-1-methyl-1H-indole-2-carboxamide

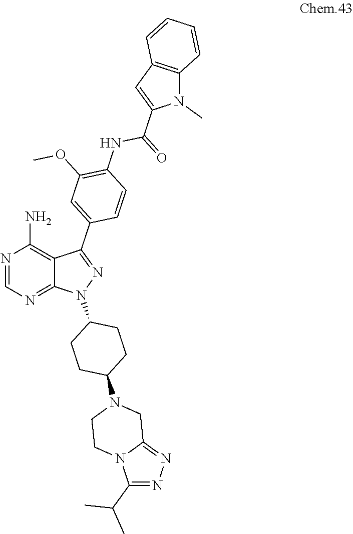 [SMILES: COc1cc(-c2nn([C@H]3CC[C@H](N4CCn5c(nnc5C(C)C)C4)CC3)c3ncnc(N)c23)ccc1NC(=O)c1cc2ccccc2n1C]

RK-20920:

N-(4-(4-amino-7-((1S,4S)-4-(3-methyl-5,6-dihydroimidazo[1,5-a]pyrazin-7(8H)-yl)cyclohexyl)-7H-pyrrolo [2,3-d]pyrimidin-5-yl)-2-methoxyphenyl)-1-methyl-1H-indole-2 -carboxamide

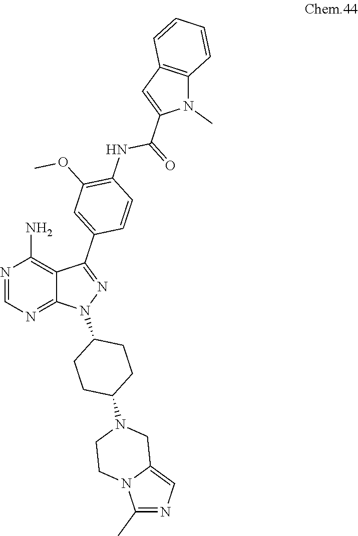 [SMILES: COc1cc(-c2nn([C@H]3CC[C@@H](N4CCn5c(cnc5C)C4)CC3)c3ncnc(N)c23)ccc1NC(=O)c1cc2ccccc2n1C]

RK-20921:

N-(4-(4-amino-7-((1R,4R)-4-(3-methyl-5,6-dihydroimidazo[1,5-a]pyrazin-7(8H)-yl)cyclohexyl)-7H-pyrrolo[2,3-d]pyrimidin-5-yl)-2-methoxyphenyl)-1-methyl-1H-indole-2-carboxamide

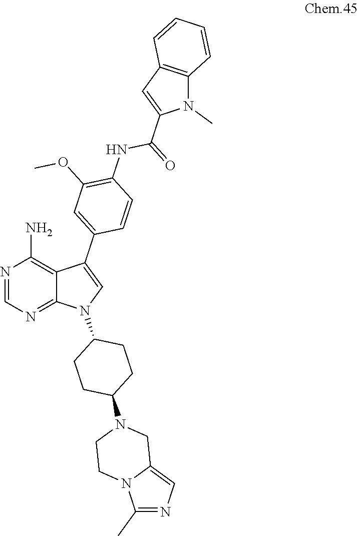 [SMILES: COc1cc(-c2cn([C@H]3CC[C@H](N4CCn5c(cnc5C)C4)CC3)c3ncnc(N)c23)ccc1NC(=O)c1cc2ccccc2n1C]

RK-20930:

N-(4-(4-amino-7-((1S,4S)-4-(2-methyl-6,7-dihydropyrazolo[1,5-a]pyrazin-5(4H)-yl) cyclohexyl)-7H-pyrrolo[2,3-d]pyrimidin-5-yl)-2-methoxyphenyl)-1-methyl-1H-indole-2-carboxamide

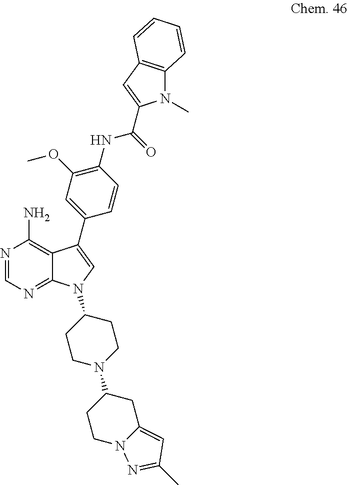

RK-20932:

N-(4-(4-amino-7-((1S,4S)-4-(6,7-dihydropyrazolo[1,5-a]pyrazin-5(4H)-yl)cyclohexy 1)-7H-pyrrolo[2,3-d]pyrimidin-5-yl)-2-methoxyphenyl)-1-methyl-1H-indole-2-carboxamide

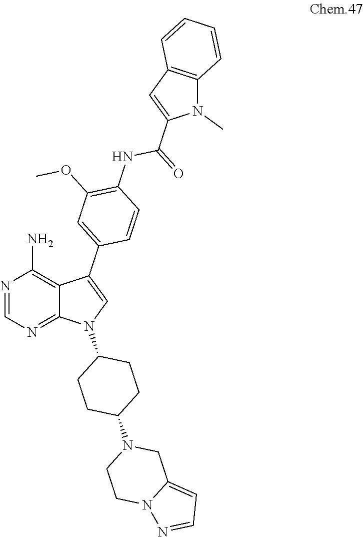 [SMILES: COc1cc(-c2cn([C@H]3CC[C@@H](N4CCn5nccc5C4)CC3)c3ncnc(N)c23)ccc1NC(=O)c1cc2ccccc2n1C]

RK-20942:

N-(4-(4-amino-7-((1R,4R)-4-(3-methyl-5,6-dihydroimidazo[1,2-a]pyrazin-7(8H)-yl)cyclohexyl)-7H-pyrrolo [2,3-d]pyrimidin-5-yl)-2-methoxyphenyl)-1-methyl-1H-indole-2-carboxamide

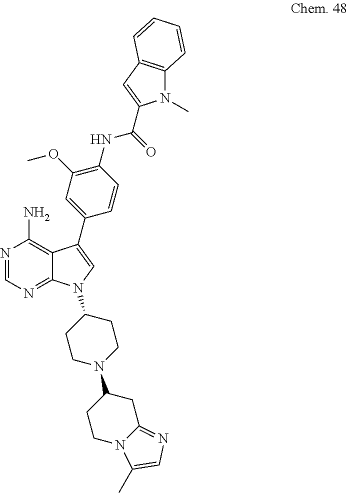

RK-20627:

7-((1R,4R)-4-(4-(2-methoxyethyl)piperazin-1-yl)cyclohexyl)-5-(4-phenoxyphenyl)-7-pyrrolo[2,3-d]pyrimidin-4-amine

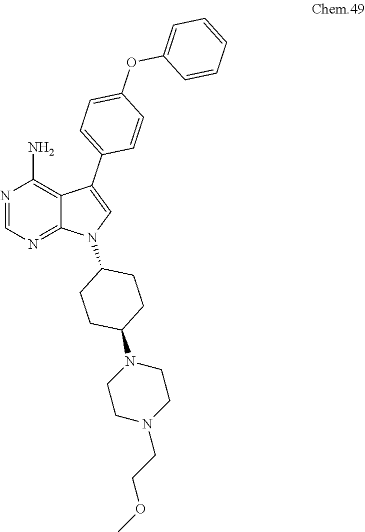 [SMILES: COCCN1CCN([C@H]2CC[C@H](n3cc(-c4ccc(Oc5ccccc5)cc4)c4c(N)ncnc43)CC2)CC1]

RK-20629:

2-(4-((1R,4R)-4-(4-amino-5-(4-phenoxyphenyl)-7H-pyrrolo[2,3-d]pyrimidin-7-yl)cyclohexyl)piperazin-1-yl)ethanol

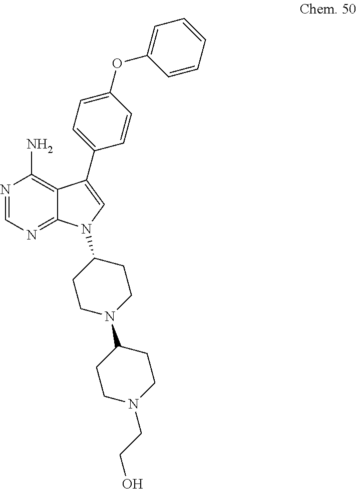

RK-20640:

7-((1R,4R)-4-(4-isopropylpiperazin-1-yl)cyclohexyl)-5-(4-phenoxyphenyl)-7H-pyrrolo[2,3-d]pyrimidin-4-amine

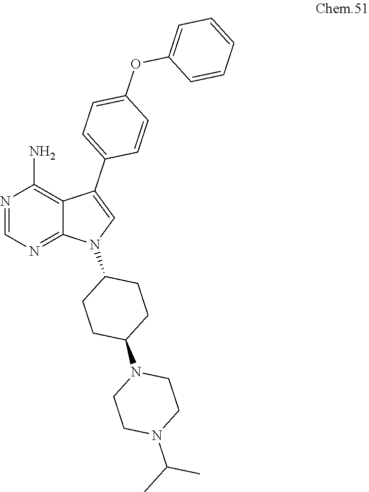 [SMILES: CC(C)N1CCN([C@H]2CC[C@H](n3cc(-c4ccc(Oc5ccccc5)cc4)c4c(N)ncnc43)CC2)CC1]

RK-20695:

5-(4-phenoxyphenyl)-7-((1R,4R)-4-((pyridin-4-ylmethyl)amino)cyclohexyl)-7H-pyrrolo[2,3-d]pyrimidin-4-amine

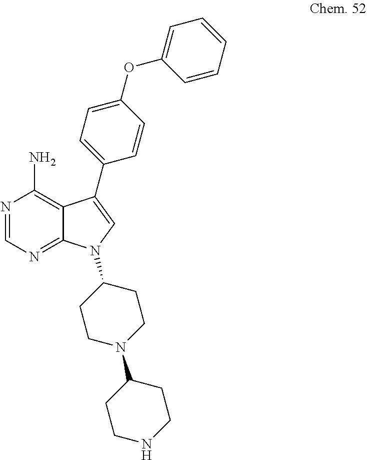

RK-20697:

5-(4-phenoxyphenyl)-7-((1R,4R)-4-((pyridin-3-ylmethyl)amino)cyclohexyl)-7H-pyrrolo[2,3-d]pyrimidin-4-amine

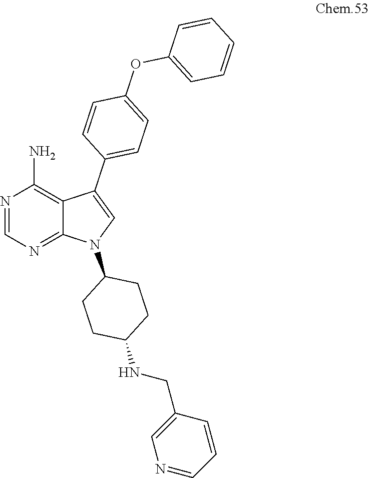 [SMILES: Nc1ncnc2c1c(-c1ccc(Oc3ccccc3)cc1)cn2[C@H]1CC[C@H](NCc2cccnc2)CC1]

RK-20698:

5-(4-phenoxyphenyl)-7-((1S,4S)-4-((pyridin-2-ylmethyl)amino)cyclohexyl)-7H-pyrrolo[2,3-d]pyrimidin-4-amine

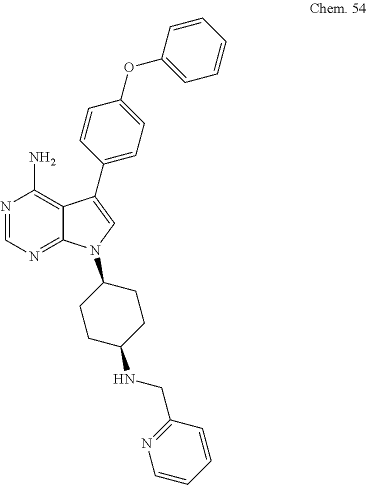 [SMILES: Nc1ncnc2c1c(-c1ccc(Oc3ccccc3)cc1)cn2[C@H]1CC[C@@H](NCc2ccccn2)CC1]

RK-20710:

7-((1S,4S)-4-(((1-methyl-1H-pyrazol-5-yl)methyl)amino)cyclohexyl)-5-(4-phenoxyphenyl)-7H-pyrrolo[2,3-d]pyrimidin-4-amine

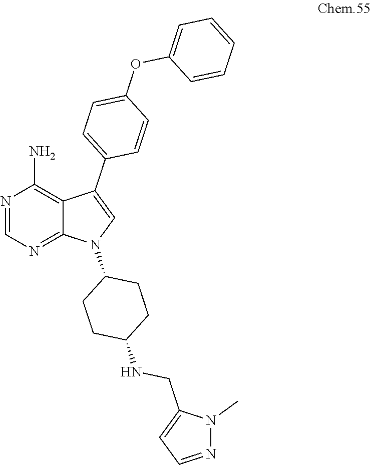 [SMILES: Cn1nccc1CN[C@H]1CC[C@@H](n2cc(-c3ccc(Oc4ccccc4)cc3)c3c(N)ncnc32)CC1]

RK-20711:

7-((1R,4R)-4-(((1-methyl-1H-pyrazol-5-yl)methyl)amino)cyclohexyl)-5-(4-phenoxyphenyl)-7H-pyrrolo[2,3-d]pyrimidin-4-amine

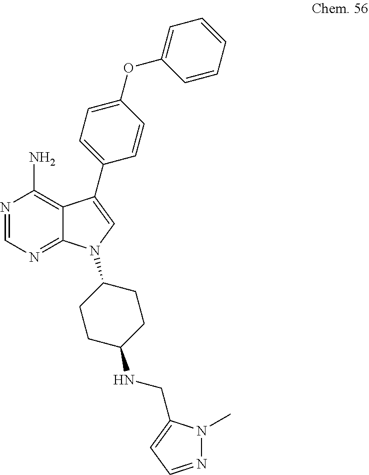 [SMILES: Cn1nccc1CN[C@H]1CC[C@H](n2cc(-c3ccc(Oc4ccccc4)cc3)c3c(N)ncnc32)CC1]

RK-20712:

7-((1S,4S)-4-(((1-methyl-1H-pyrazol-3-yl)methyl)amino)cyclohexyl)-5-(4-phenoxyphenyl)-7H-pyrrolo[2,3-d]pyrimidin-4-amine

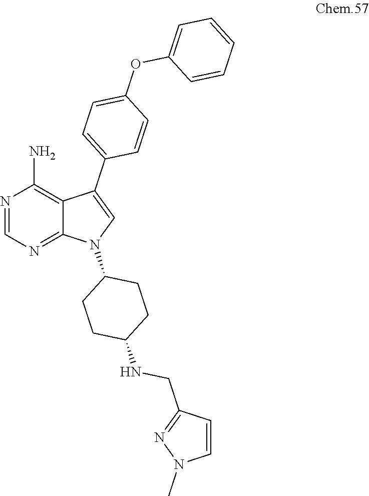 [SMILES: Cn1ccc(CN[C@H]2CC[C@@H](n3cc(-c4ccc(Oc5ccccc5)cc4)c4c(N)ncnc43)CC2)n1]

RK-20724:

1-((1S,4S)-4-(8-methyl-3,8-diazabicyclo[3.2.1]octan-3-yl)cyclohexyl)-3-(4-phenoxyphenyl)-1H-pyrazolo[3,4-d]pyrimidin-4-amine

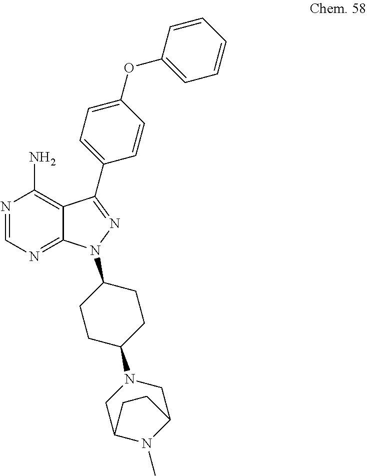 [SMILES: CN1C2CCC1CN([C@H]1CC[C@@H](n3nc(-c4ccc(Oc5ccccc5)cc4)c4c(N)ncnc43)CC1)C2]

RK-20733:

(S)-3-(((1S,4R)-4-(4-amino-5-(4-phenoxyphenyl)-7H-pyrrolo[2,3-d]pyrimidin-7-yl)cyclohexyl)amino)pyrrolidin-2-one

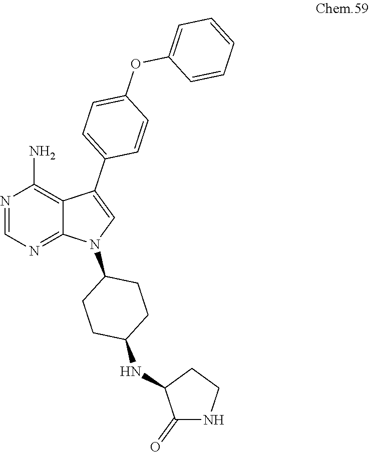 [SMILES: Nc1ncnc2c1c(-c1ccc(Oc3ccccc3)cc1)cn2[C@H]1CC[C@@H](N[C@H]2CCNC2=O)CC1]

RK-20734:

(S)-3-(((1R,4S)-4-(4-amino-5-(4-phenoxyphenyl)-7H-pyrrolo[2,3-d]pyrimidin-7-yl)cyclohexyl)amino)pyrrolidin-2-one

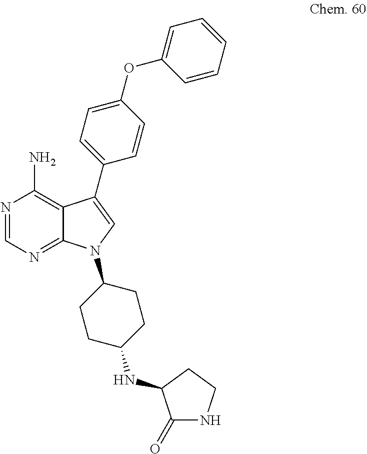 [SMILES: Nc1ncnc2c1c(-c1ccc(Oc3ccccc3)cc1)cn2[C@H]1CC[C@H](N[C@H]2CCNC2=O)CC1]

RK-20758:

7-((1S,4S)-4-((8-methyl-8-azabicyclo[3.2.1]octan-3-yl)amino)cyclohexyl)-5-(4-phenoxyphenyl)-7H-pyrrolo[2,3-d]pyrimidin-4-amine

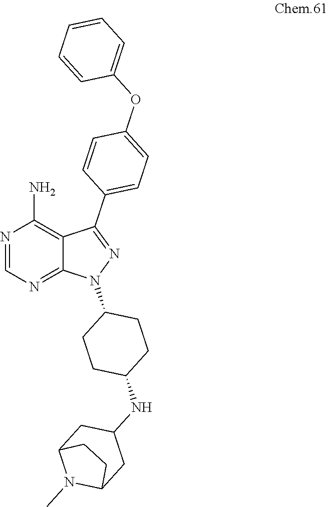 [SMILES: CN1C2CCC1CC(N[C@H]1CC[C@@H](n3nc(-c4ccc(Oc5ccccc5)cc4)c4c(N)ncnc43)CC1)C2]

RK-20898:

5-(4-phenoxyphenyl)-7-((1R,4R)-4-(3-(trifluoromethyl)-5,6-dihydro-[1,2,4]triazolo[4,3-a]pyrazin-7(8H)-yl)cyclohexyl)-7H-pyrrolo[2,3-d]pyrimidin-4-amine

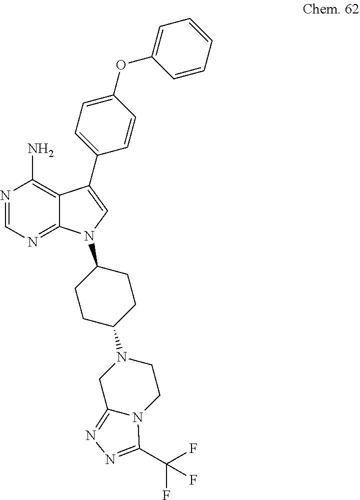 [SMILES: Nc1ncnc2c1c(-c1ccc(Oc3ccccc3)cc1)cn2[C@H]1CC[C@H](N2CCn3c(nnc3C(F)(F)F)C2)CC1]

RK-20620:

1-((1R,4R)-4-(4-methylpiperazin-1-yl)cyclohexyl)-3-(4-phenoxyphenyl)-1H-pyrazolo[3,4-d]pyrimidin-4-amine

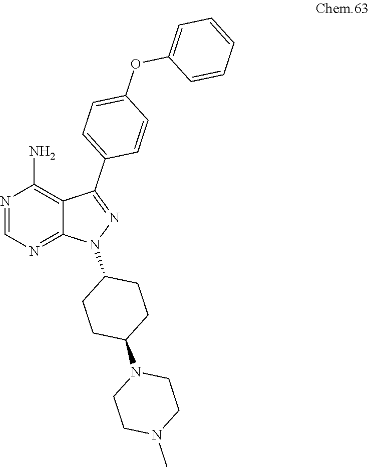 [SMILES: CN1CCN([C@H]2CC[C@H](n3nc(-c4ccc(Oc5ccccc5)cc4)c4c(N)ncnc43)CC2)CC1]

The following compounds are commercially available:

AC220 (also known as quizartinib):

Urea, N-

[5-(1,1-dimethylethyl)-3-isoxazolyl]-N′-[4-[7-[2-(4-morpholinyl)ethoxy]imidazo[2,1-b]benzothiazol-2-yl]phenyl]-.

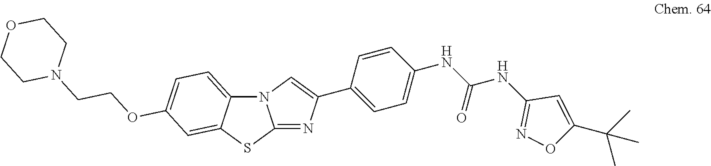 [SMILES: CC(C)(C)c1cc(NC(=O)Nc2ccc(-c3cn4c(n3)sc3cc(OCCN5CCOCC5)ccc34)cc2)no1]

ABT-199 (also known as venetoclax):

4-[4-[2-(4-chlorophenyl)-4,4-dimethyl-1-cyclohexen-1-yl]methyl]-1-piperazinyl]-N-[[3-nitro-4-[[(tetrahydro-2H-pyran-4-yl)methyl]amino]phenyl]sulfonyl]-2-(1H-pyrrolo[2,3-b]pyridin-5-yloxy)-benzamide

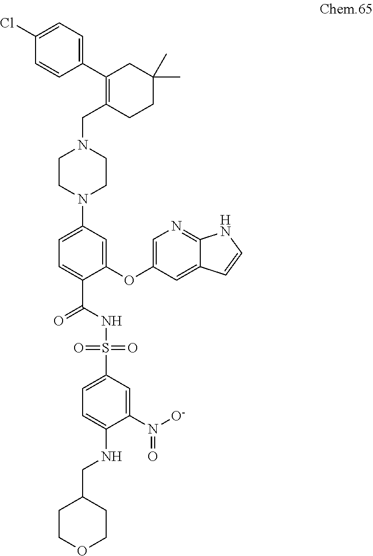

### Human Samples

All experiments were performed with authorization from the Institutional Review Board for Human Research at RIKEN and Toranomon Hospital. All patient samples were collected at Toranomon Hospital with written informed consent. CB samples were purchased from Lonza.

Mice

NOD.Cg-PrkdcscidIl2rgtmlWjl/Sz (NSG) mice were developed at The Jackson Laboratory (Shultz LD et al., J Immunol 2005, Ishikawa F et al., Blood 2005) . Mice were bred and maintained under defined flora at the animal facility of RIKEN and at The Jackson Laboratory according to guidelines established by at the Institutional Animal Committees at each institution.

### Flow Cytometry

The following monoclonal antibodies (mAbs) were used for flow cytometry: Mouse anti-human CD19, CD3, CD33, CD34, CD38, CD4, and CD45; Rat anti-mouse Ter119 and CD45 (BD Biosciences). Analyses were performed with FACSAriaIII and FAC-SCantoII (BD). To obtain cells for xenogeneic transplantation, BV786-conjugated anti-CD3 mAb, BV605-conjugated anti-CD19 mAb, BV421-conjugated anti-CD33 mAb, PE-Cy7-conjugaed anti-CD34 mAb, APC-conjugated anti-CD38 mAb, FITC-conjutaged anti-CD90 mAb, and PE-conjugated anti-CD45RA mAb were used. For single cell sorting, 100mum nozzle was used.

### Transplantation

NSG newborns received 1.5Gy total body irradiation followed by intravenous injection of purified human cells. Donor cells were purified according to their cell surface phenotype using monoclonal antibodies against human CD34, CD38, CD90, CD45RA, CD3, CD19 and CD33. Engraftment levels of human cells in the NSG recipients were assessed by retro-orbital phlebotomy and flow cytometry.

### Genome Analysis

DNA was extracted from human cells purified from patient samples or recipient organs using DNeasy Blood & Tissue Kit (QIAGEN). PCR detection of FLT3−ITD was performed using TaKaRa PCR FLT3/ITD Mutation Detection Set (Takara). The DNA sequences in bulk were determined by next-generation DNA sequencing (NGS). After fragmented with a Covaris 5220 (Covaris Inc., Wobum, Mass. USA), the fragmented genomic DNA (10 ng) was converted to NGS sequencing library with a KAPA Hyper Prep Kit (KAPA Biosystems, Wilmington, Mass.) according to protocol provided by the supplier. Targeted sequencing of AML-related genes was carried out by a hybridization-capture method with xGen AML Cancer Panel v1.0 (Integrated DNA Technologies, Inc., Coralville, Iowa) according to protocol provided by the supplier. The hybridization-captured DNA library was subjected to NGS in a paired-end read mode (600 cycles) with an Illumina Miseq (Illumina, Inc., San Diego, Calif.). The obtained DNA sequences were mapped onto human genome sequence (hg19) using BWA, then realigned with a Realigner Target Creator in Geome Analysis Toolkit. After treatment with Fix Mate Information in Picard and Quality Score Covariate and Table Recalibration in Genome Analysis Toolkit (v.1.6-13), variants were detected with VarScan.

Single-cell variation analysis was carried out for single cells sorted on a BD FACS Aria into 96-well plates. After single-cell genome amplification with a MALBAC Single Cell WGA Kit (Yikon Genomics, Beijin, China), target regions of genes of interest were PCR-amplified with primers including well indexes by PCR as described in Supplementary Information. The PCR products were sequenced in a paired-end read mode (600 cycles) on an Illumina Miseq. The obtained DNA sequences were mapped onto human genome sequence (hg 19) with BWA (v0.7.12) and merged paired-end reads with SAMtools. Variant detection and frequency calculation were done with mpileup in SAMtools.

### In Vivo Treatment

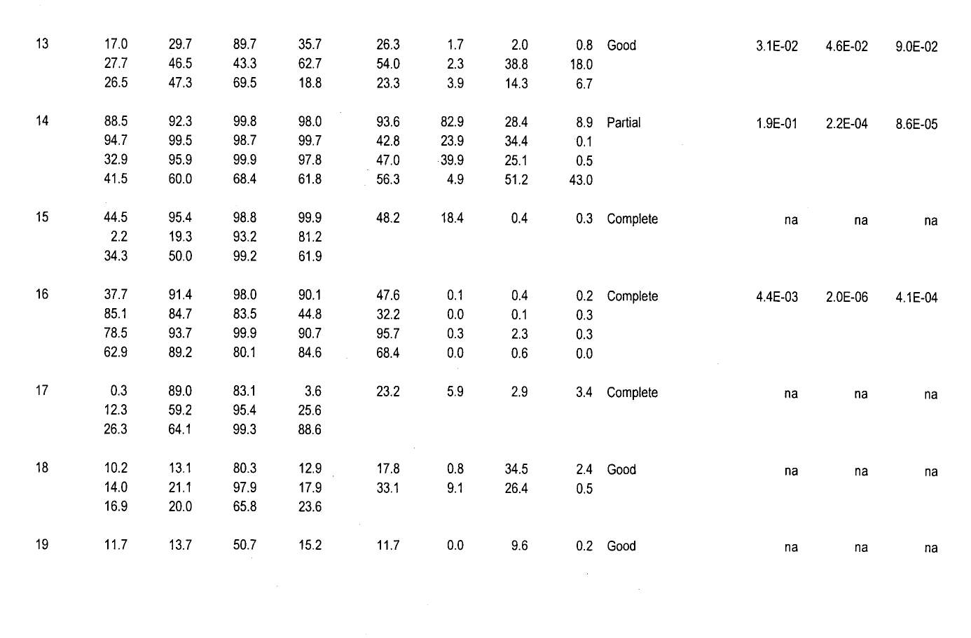

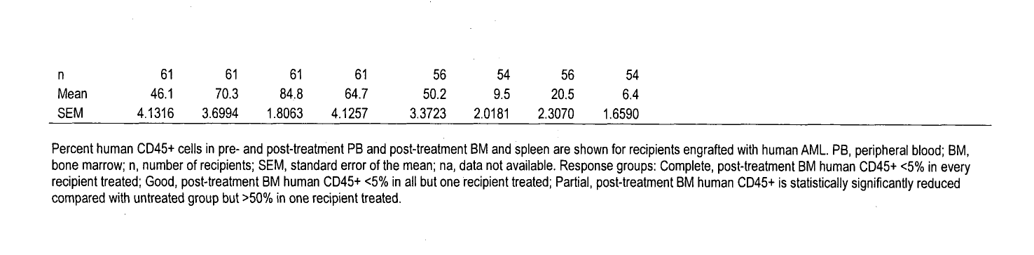

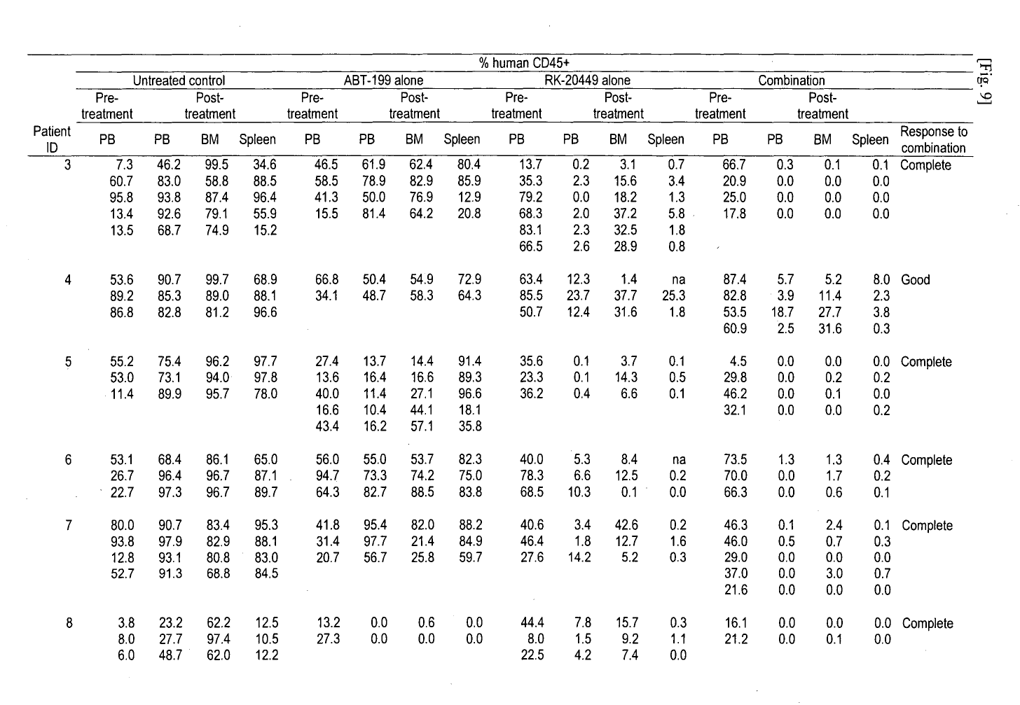

In vivo treatment experiments were performed with AML-engrafted NSG recipients using RK-20449 (Ref. STM 2013) and ABT-199 (Souers A J et al., Nature Medicine 2013, Pan R et al., Cancer Discovery 2014). The recipients were administered RK-20449 intraperitoneally 30 mg/kg twice a day, ABT-199 orally 70 mg/kg once a day or both RK-20449 and ABT-199 at these dosing schedules. The mice were sacrificed when they became moribund or after 4˜6 weeks of treatment, and human AML chimerisms in BM, spleen and PB were determined using flow cytometry. In secondary transplantation, each mouse received 7-AAD(-) viable human CD45+cells from 2.5% of total BM that remained in AML-engrafted recipients at the time of sacrifice, to simulate relapse occurring from residual viable AML cells. All treated recipients and their pre- and post-treatment engraftment data are tabulated in FIGS. 8 and 9.

### Statistical Analysis

Numerical data are presented as mean+/−SEM. The differences were examined with two-tailed t test (GraphPad Prism, GraphPad).

### EXAMPLES

### Example 1

Evaluation of FLT3−ITD+leukemic subclones

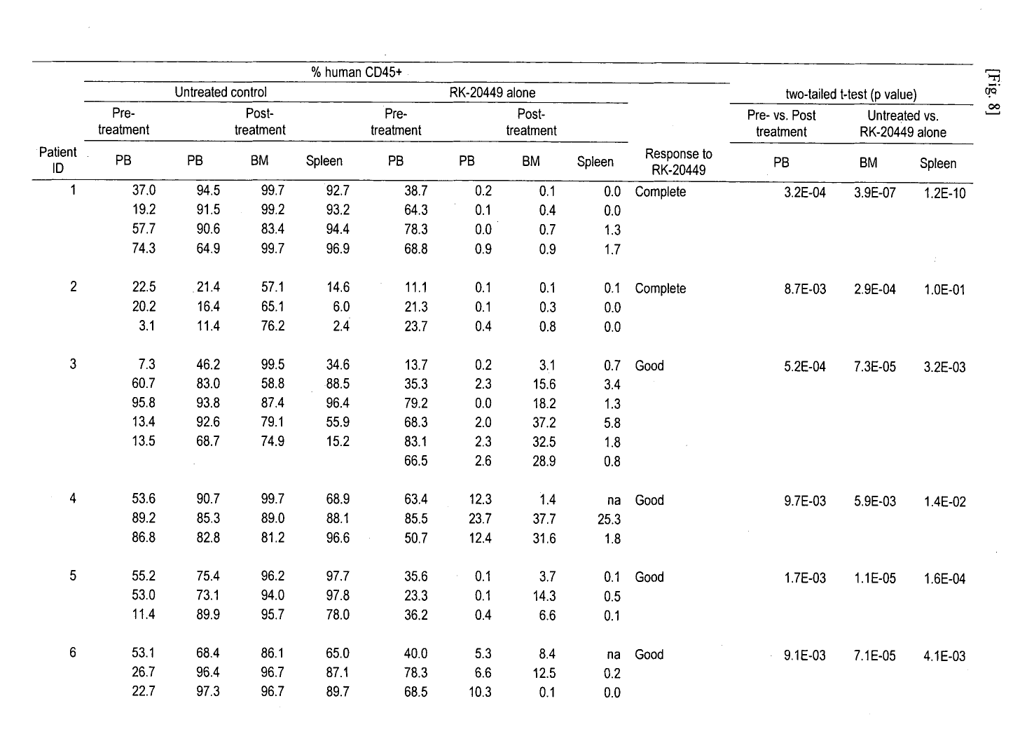

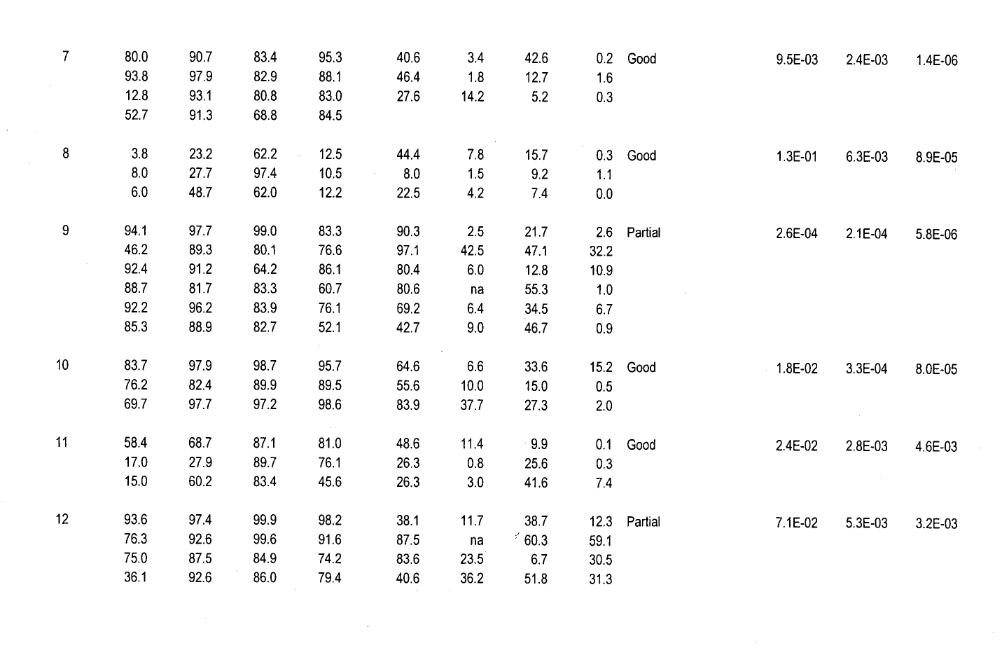

Bone marrow (BM) or peripheral blood (PB) samples were obtained from 23 patients with FLT3−ITD+AML, whose diagnosis, clinical course and clinical outcomes are detailed in FIG. 7. The majority of patients had poor prognostic factors such as complex chromosomal abnormalities in addition to FLT3−ITD mutation and/or had known aggressive disease (induction failure, relapse after multiple stem cell transplantations).

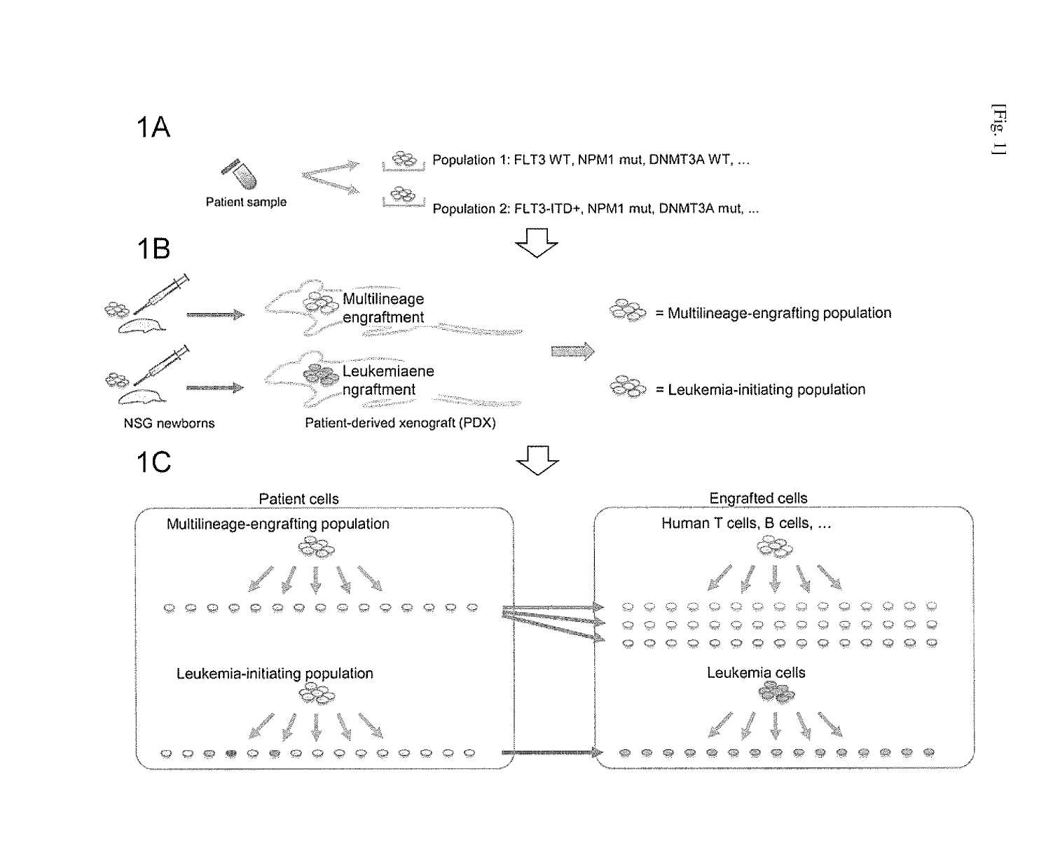

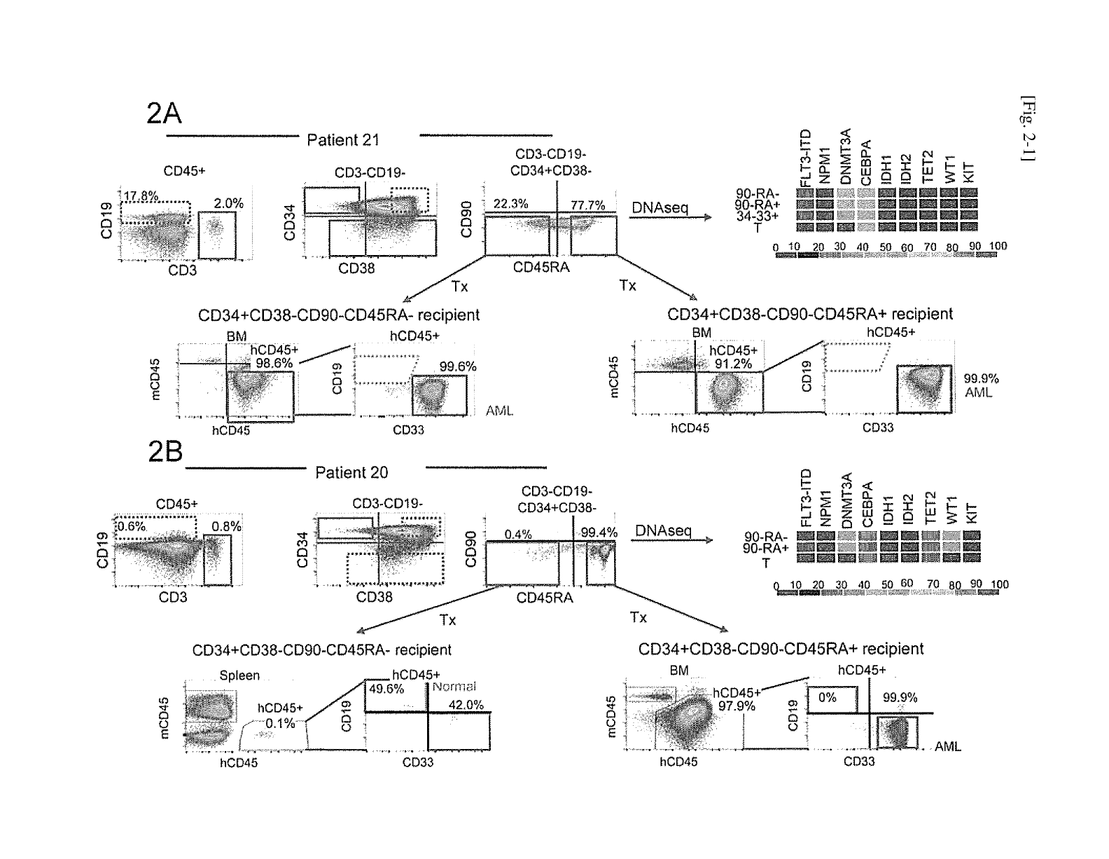

To evaluate the contribution of AML-associated somatic mutations to leukemogenesis by linking mutations with in vivo function, the following experiments performed: 1) Population-level mutation screening in surface phenotype-defined hematopoietic cell subpopulations isolated from patients, 2) Functional assessment of the in vivo fates of those subpopulations by NSG xenotransplantation, and 3) Single cell sequencing to track patient-derived clones in xenotransplantation recipients (see, FIGS. 1A, 1B, and 1C).

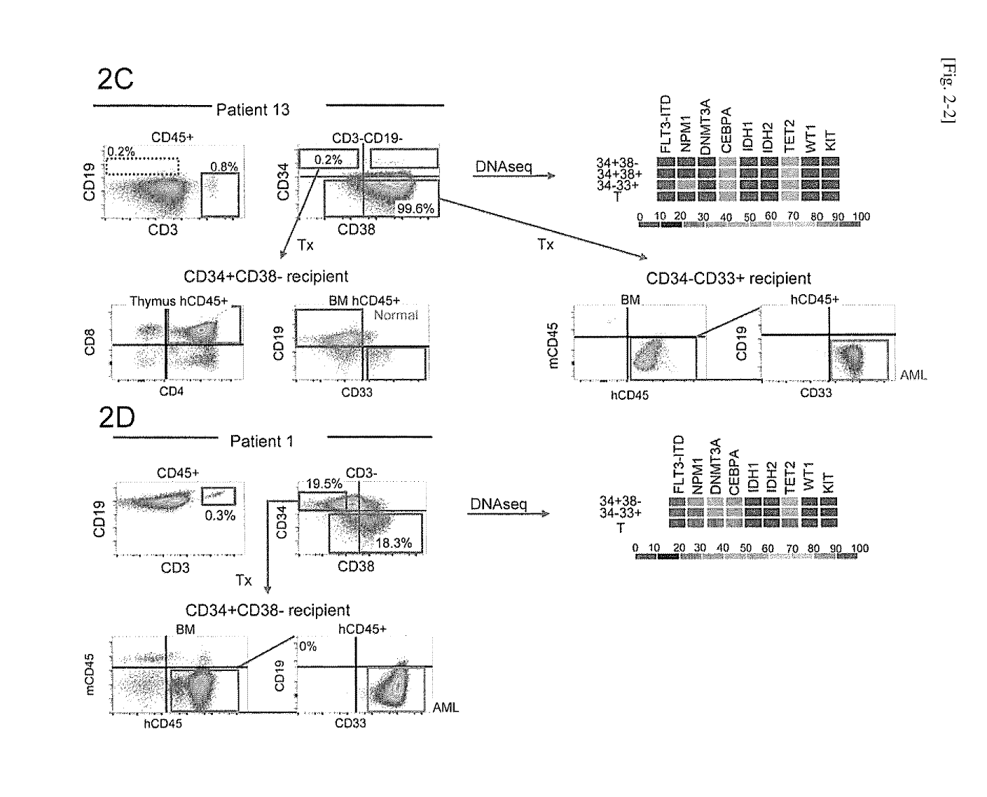

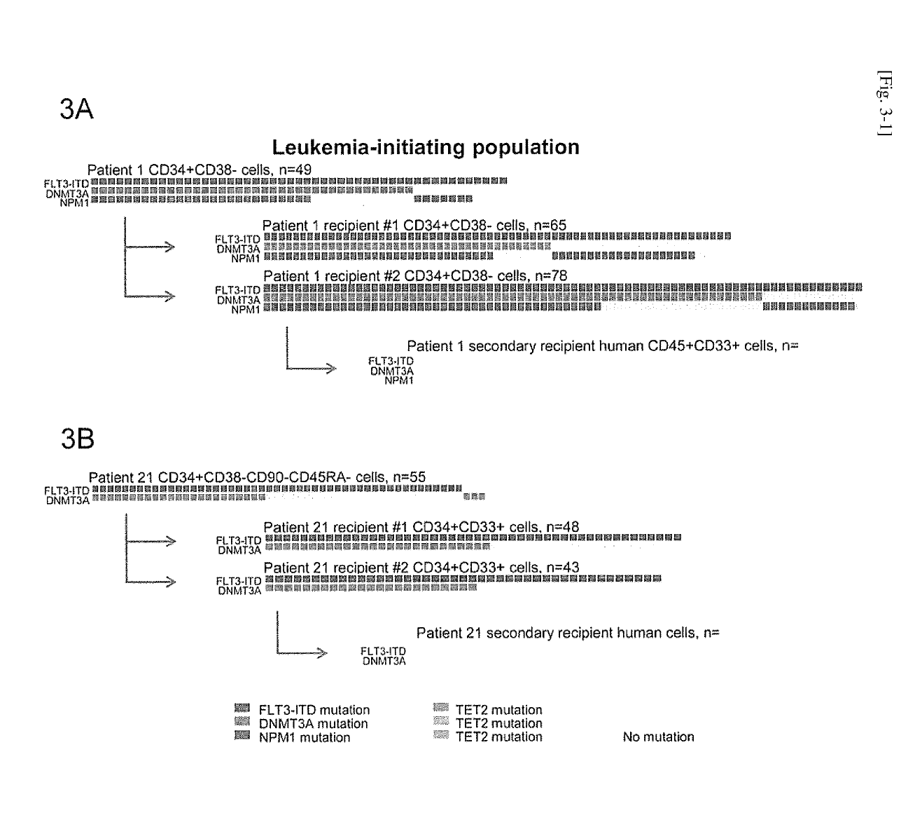

In normal human hematopoiesis, HSC-enriched CD34+CD38−cells repopulate NSG recipients. Within this population, CD34+CD38−(CD90−)CD45RA−cells are capable of multilineage human hematopoietic repopulation (see, FIG. 7). This capacity is lost as the cells differentiate, marked by acquisition of CD45RA expression followed by CD38 expression and loss of CD34 expression. These results show that surface phenotypes of the corresponding markers varied widely among patients (Representative data from four patients are shown in FIG. 2). Population-level sequencing showed patient-to-patient variation in mutations as expected. However, there were distinct differences in frequencies of mutated alleles among populations with different cell surface phenotypes.

The transplanted patient-derived subpopulations were sorted according to surface phenotypes into NSG mice. For example, in Patient 21 (FIG. 2A) and Patient 20 (FIG. 2B), CD34+CD38−subpopulation was further subdivided into CD90−CD45RA− and CD90−CD45RA+subpopulations, corresponding to primitive HSCs and more differentiated HPCs in normal human hematopoiesis. Depending on the patient, subpopulations with similar surface phenotypes showed distinct in vivo fates through NSG xenotransplantation. CD34+CD38−CD90−CD45RA−HSC-phenotype subpopulation from Patient 21 initiated AML in vivo while those from Patient 20 showed multilineage engraftment. CD34+CD38−HSC/HPC-phenotype subpopulation from Patient 13 (FIG. 2C) showed multilineage engraftment while those from Patient 1 (FIG. 2D) initiated AML in vivo. In Patients 20 and 13, CD34+CD38−CD90−CD45RA+progenitor-phenotype and CD34−CD33+mature myeloid-phenotype subpopulation contained LICs, respectively. Presence and absence of FLT3−ITD mutation correlated with leukemia-initiation and multilineage engraftment, respectively, while other mutations did not segregate with in vivo cell fates.

Between patients, cells of the same surface phenotype nonetheless showed distinct behaviors in vivo. For instance, CD34+CD38−CD90−CD45RA−population from Patient 21 initiated AML in NSG mice and therefore contained leukemia-initiating cells (LICs). On the other hand, population with the same phenotype population from Patient 20 reconstituted multilineage human hematopoiesis in NSG mice and therefore contained multilineage-engrafting hematopoietic stem cells. Similarly, Patient 13-derived CD34+CD38−population was a multilineage-engrafting hematopoietic stem cell-containing population, while Patient 1-derived CD34+CD38−population was a leukemia-initiating cell-containing population.

Historically, differences in hematopoietic repopulation function have been attributed to cell surface phenotype, yet here, populations with the same surface phenotype possessed different repopulating function between patients. Thus, mutational profiling was performed at the single cell level in each population as clonal diversity within a population with multiple mutations may not be characterized accurately by bulk allelic frequencies alone. Clonal structures within those subpopulations were defined and mutation(s) associated with leukemia-initiating function versus multilineage-engrafting function were identified at the single cell level.

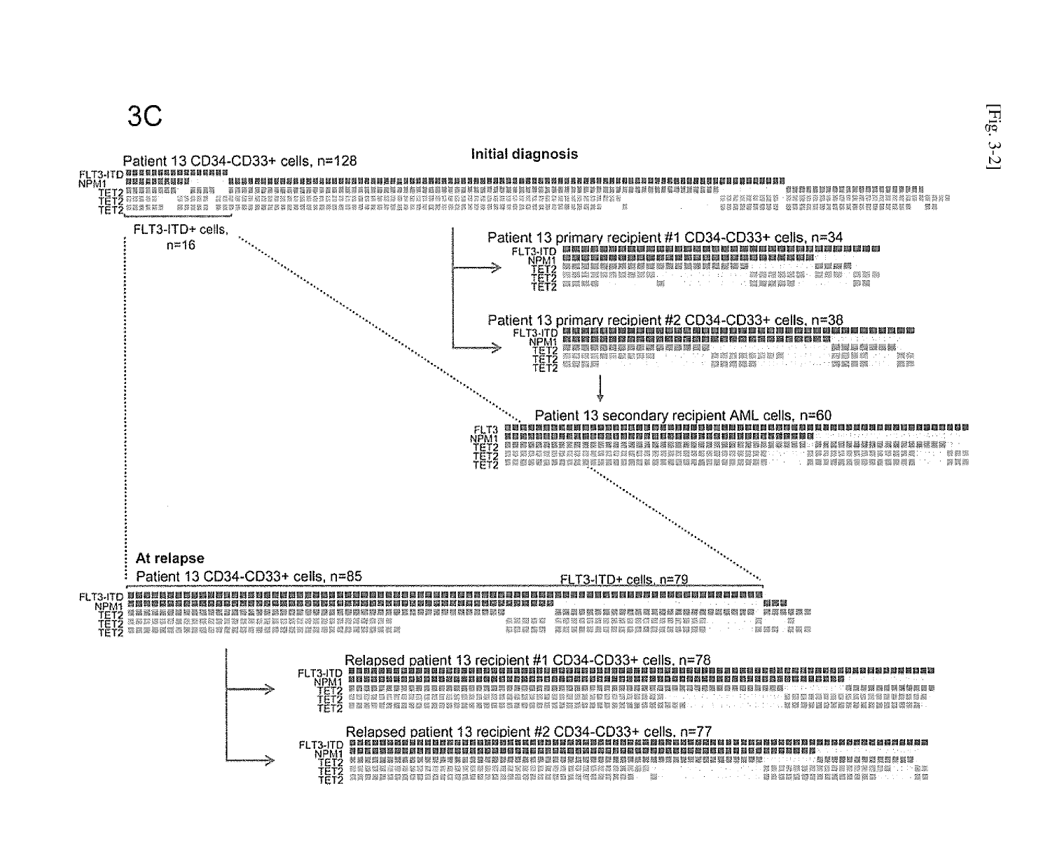

Surface phenotype-defined leukemia-initiating cell-containing populations and multilineage-engrafting stem cell-containing populations isolated from patients consisted of individual cells with diverse combinations of mutations (see, FIGS. 3A and 3B). For example, Patient 21-derived CD34+CD38−CD90−CD45RA−single cells contained four distinct subclones: FLT3−ITD+/DNMT3A-mutant, FLT3−ITD+/DNMT3A−WT, FLT3 WT/DNMT3A-mutant or FLT3−WT/DNMT3A−WT (FIG. 3A). In addition, patient-to-patient variability in frequencies of mutations and their combinations in individual cells were observed among both leukemia-initiating cell-containing and multilineage-engrafting stem cell-containing populations. Every cell in Patient 1 leukemia-initiating population was FLT3−ITD+ whereas only 16 of 128 single cells were FLT3−ITD+ in Patient 13 leukemia-initiating population (FIG. 3A). Similarly, every cell in Patient 13 multilineage-engrafting stem cell population was FLT3−WT whereas 18 of 117 single cells were FLT3−ITD+ in Patient 20 multilineage-engrafting stem cell population (FIG. 3B). These findings demonstrate that human AML cell subpopulations consisting of subclones with diverse mutational profiles have distinct in vivo cell fates (leukemia-initiating or multilineage-engrafting) when transplanted into NSG recipients.

AML patient-derived leukemic and multilineage hematopoietic cells engrafted in NSG recipients were also evaluated. By profiling mutations in individual engrafted human leukemic cells and human multilineage hematopoietic cells, leukemia-initiating clones and pre-leukemic stem cell clones were identified among various subclones present in patient-derived leukemia-initiating cell-containing and multilineage-engrafting populations, respectively. Subsequently, by comparing mutations present in individual clones with known in vivo cell fates, mutations that contribute to leukemia-initiation were determined. Surprisingly, in three cases (Patients 1, 13 and 21), every human cell in recipients transplanted with leukemia-initiating population was FLT3−ITD+, regardless of the frequency of FLT3−ITD+single cells or the cell surface phenotype of the parental leukemia-initiating population. In addition, there were DNMT3A WT (Patients 1, 20 and 21), NPM1 WT (Patient 13) and TET2 WT (Patient 13) subclones among engrafted FLT3−ITD+AML cells in primary and secondary recipients, showing that FLT3−ITD is most strongly linked with leukemogenesis and other mutations are not obligatory (see, FIG. 3A).

This was not an observation limited to PDX models. In Patient 13, the frequency of FLT3−ITD+single cells in leukemia-initiating CD34−CD33+population substantially increased from initial diagnosis to relapse while NPM1 WT and TET2 WT subclones among the FLT3−ITD+subclones persisted, showing that FLT−ITD conferred the greatest malignant potential associated with relapse in the patient (see, FIG. 3A). On the other hand, every engrafted human cell in recipients transplanted with multilineage-engrafting population was FLT3 WT, even in Patient 20 whose pre-leukemic (multilineage-engrafting) population contained FLT3−ITD subclones (see, FIG. 3B). Moreover, there were DNMT3A-mutated (Patient 21), NPM1-mutated (Patient 13) and TET2-mutated (Patient 13) subclones in engrafted multilineage human hematopoietic cells in recipients, indicating that these mutations do not preclude multilineage hematopoiesis. Even single cells harboring both NPM1 and TET2 mutations differentiated into CD19+B cells (see, FIG. 3B). These findings show that FLT3−ITD mutation confers the greatest malignant potential with other mutations playing cooperative roles and strongly suggests that acquisition of the FLT3−ITD mutation confers AML-initiating capacity regardless of surface phenotype and co-existing mutations.

### Example 2

Effects of Kinase Inhibition on Human FLT3−ITD+AML Subclones with Diverse Mutations

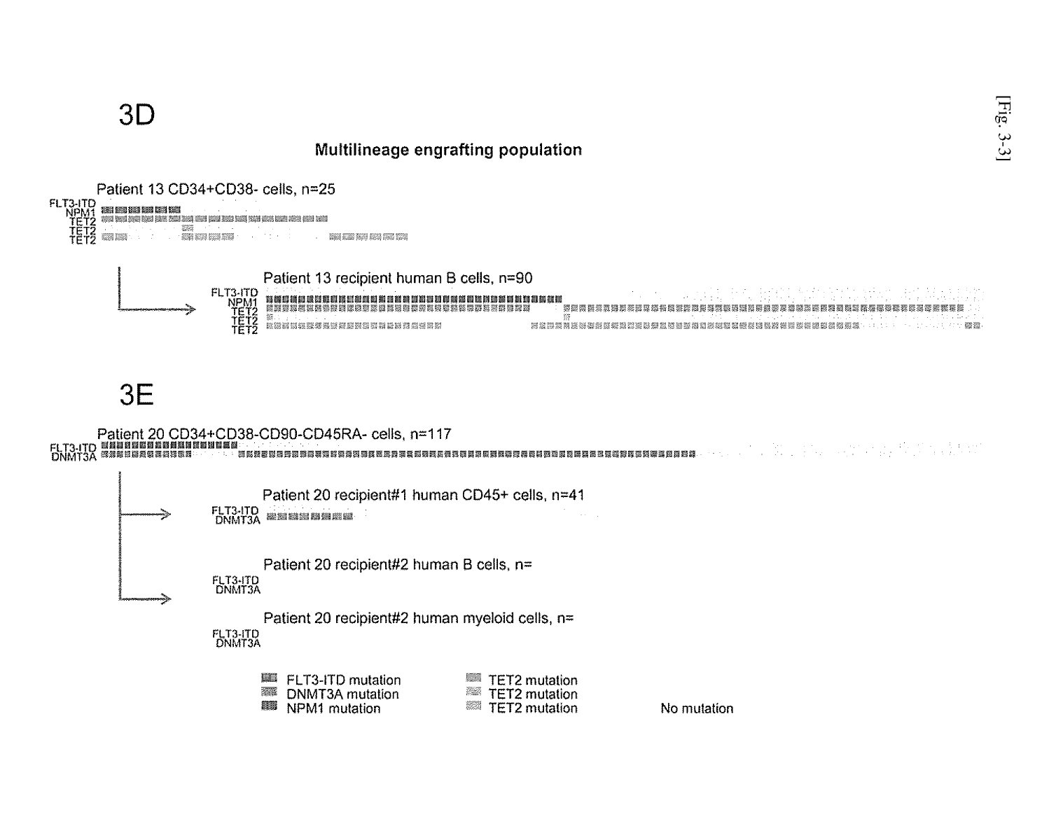

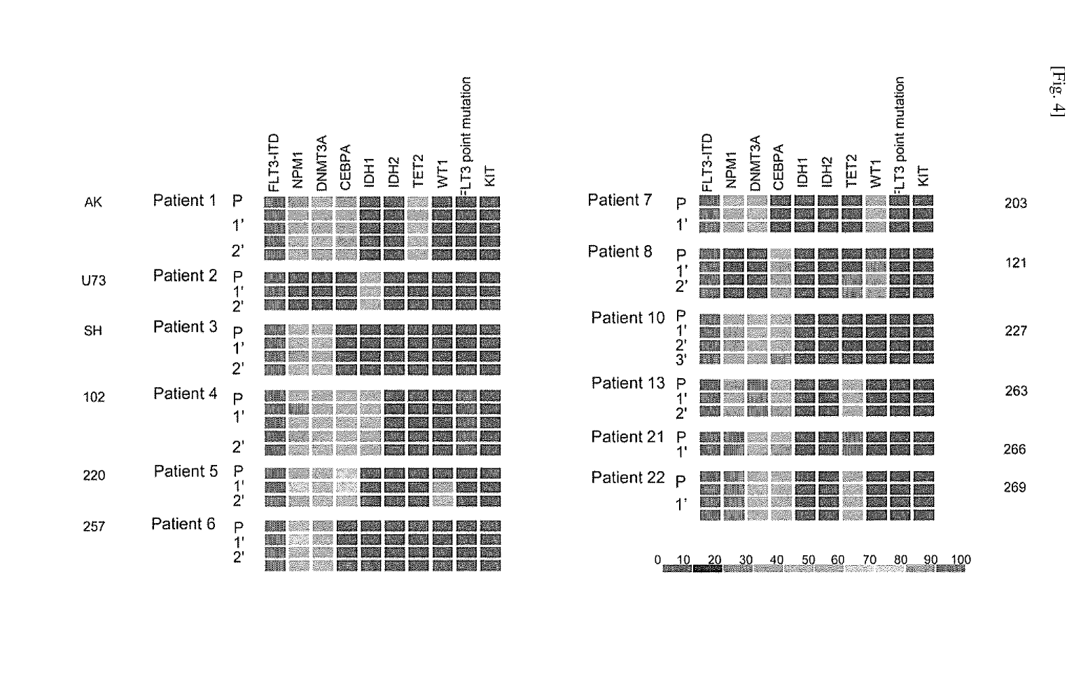

These studies examined whether inhibition of a mutated kinase alone eliminates AML in vivo using a NSG PDX model. Patient-derived cells with a FLT3−ITD mutation engraft in vivo and initiate AML, but the individual FLT3−ITD+cells harbor multiple other mutations in various combinations. These mutations may contribute to therapy responsiveness in cooperation with FLT3−ITD, and a PDX model that reflects mutational complexity of human AML cells is necessary to examine the contribution of multiple co-existing mutations. Therefore, the frequency of AML-associated somatic mutations was evaluated in the disclosed PDX model using newborn NSG xenotransplantation, and the same sets of mutations were present in patient-derived leukemia-initiating population and engrafted AML cells in recipients (see, FIG. 4). Moreover, single cell sequencing demonstrated that the NSG PDX model allowed engraftment of multiple leukemia-initiating subclones with various combinations of mutations (see, FIGS. 3A and 3B). The extent that targeting FLT3 pathway alone can result in reduction of human AML cells harboring multiple mutations in vivo was examined as follows. RK-20449 is a pyrrolo-pyrimidine-derivative multi-kinase inhibitor of Src family kinase HCK and FLT3 that effectively target human AML cells. Therefore, RK-20449 was administered to 58 NSG mice engrafted with AML from 19 FLT3−ITD+AML patients and the results are shown in FIG. 8.

In five patients, responses were complete (every recipient treated showed residual

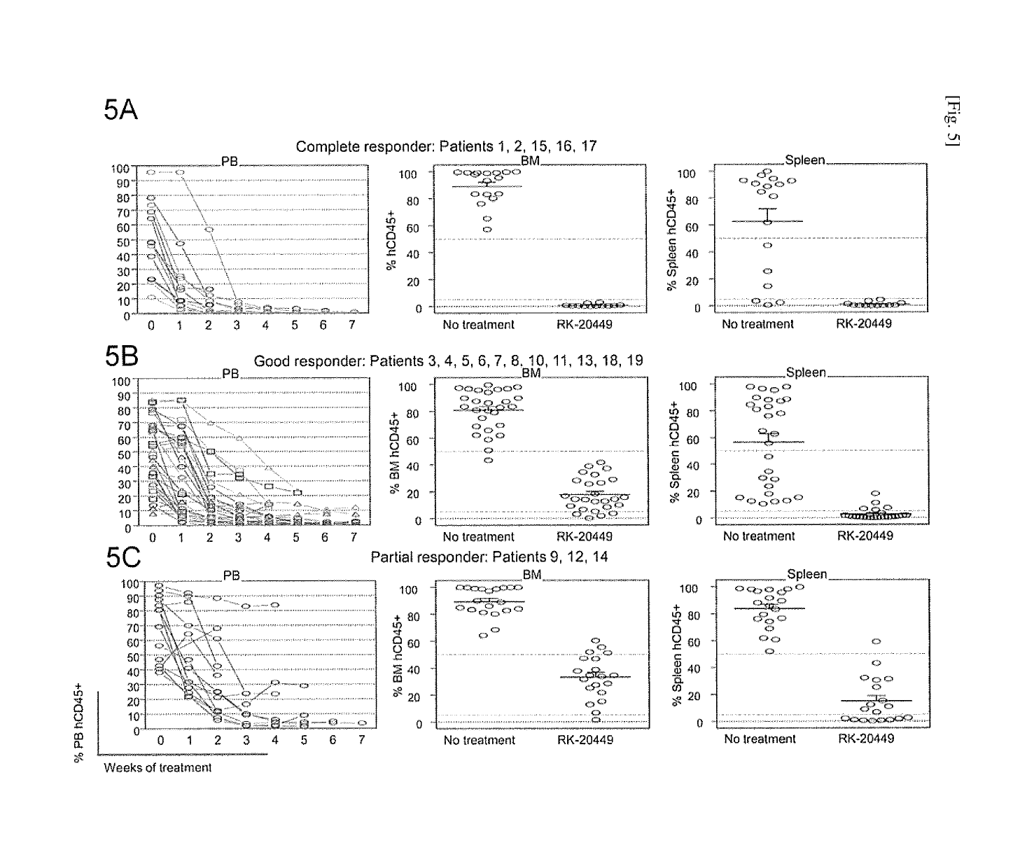

BM human CD45+chimerism <5%) (see, FIG. 5A). In these patients, RK-20449 eliminated AML cells in the BM, spleen and PB to less than 0.01% based on flow cytometry and immunohistochemistry, such as about 0.05% to about 0.01%. in all recipients treated, despite the presence of multiple mutations not directly targeted by RK-20449 (Patient 1: DNMT3A, CEBPA, TET2; Patient 2: IDH1; Patient 15: CEBPA, n-Ras; Patient 16: WT1). In 11 additional cases, RK-20449 alone resulted in complete responses in the spleen in a majority of recipients tested (see, FIG. 5B). Overall, in all 19 cases tested, FLT3−ITD+human AML cells show significant responses to single agent RK-20449 in vivo; however, residual AML cells were observed in the BM of at least one treated mouse in 14 of 19 cases (see, FIGS. 5C and 5D). Therefore, mechanisms for multi-kinase inhibition resistance in FLT3−ITD+AML cells were evaluated.

### Example 3

Effect of Combined Inhibition of Kinase and Anti-Spoptosis Pathways on FLT3−ITD+AML Cells in Vivo

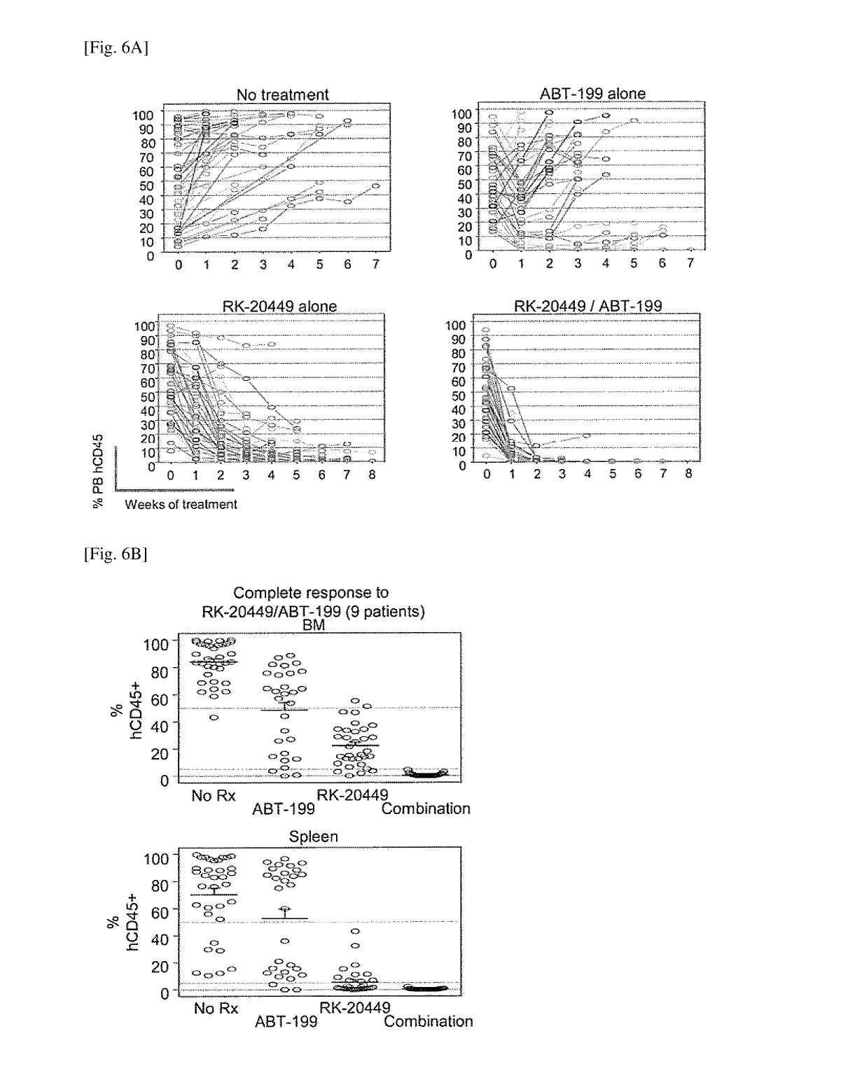

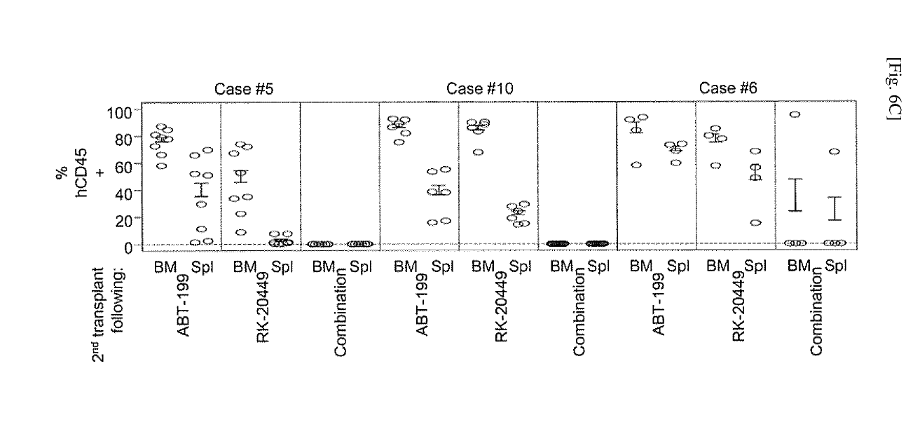

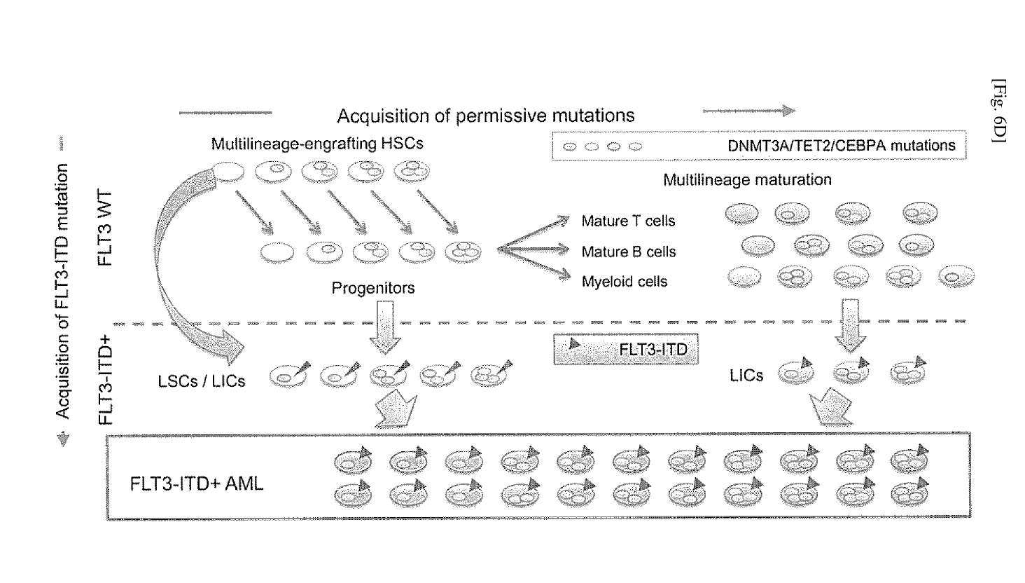

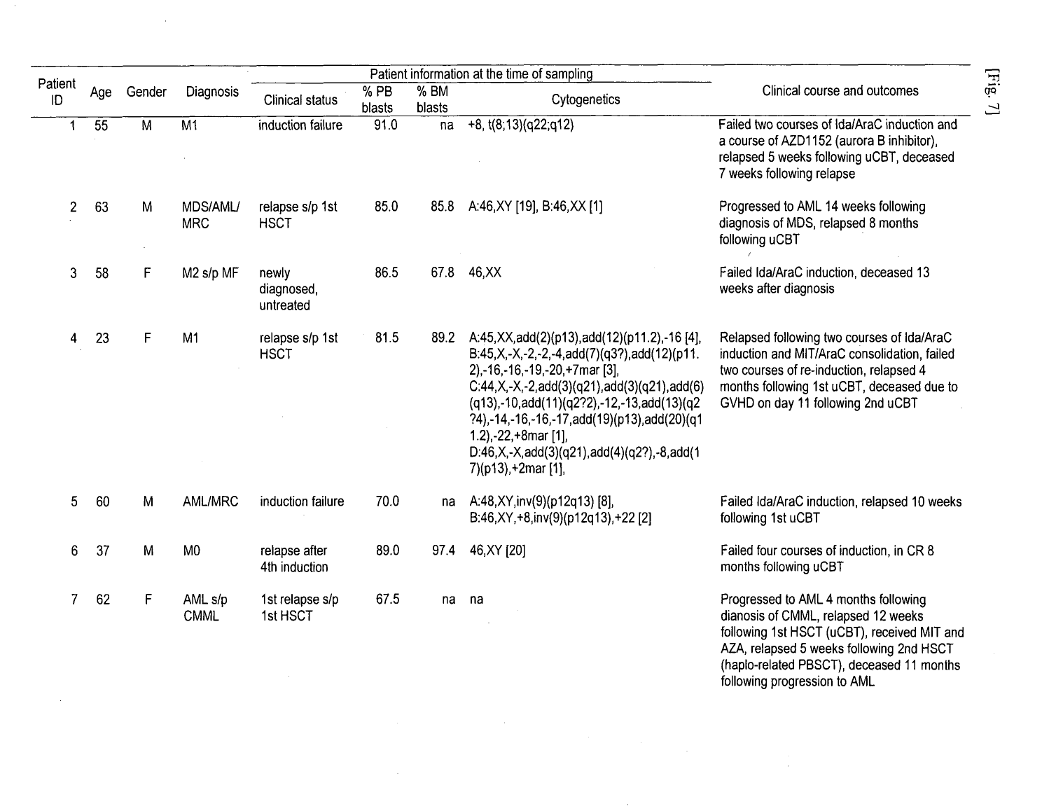

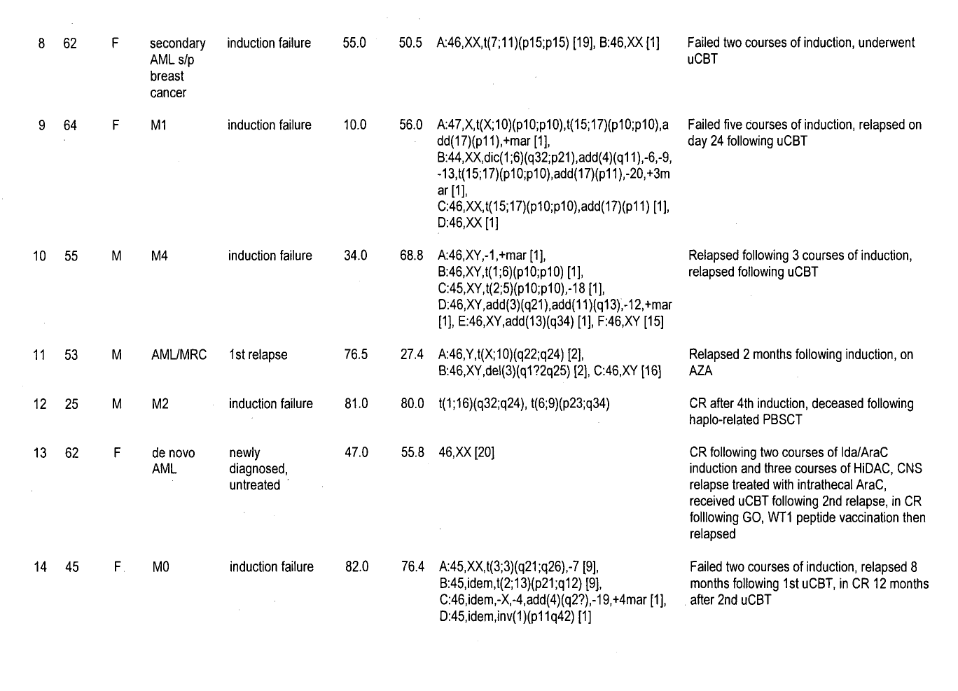

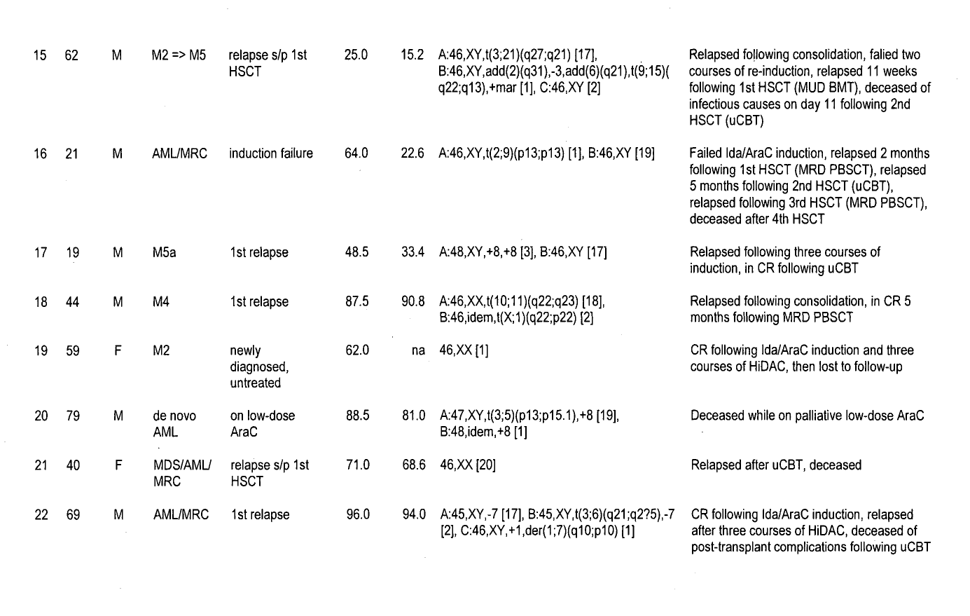

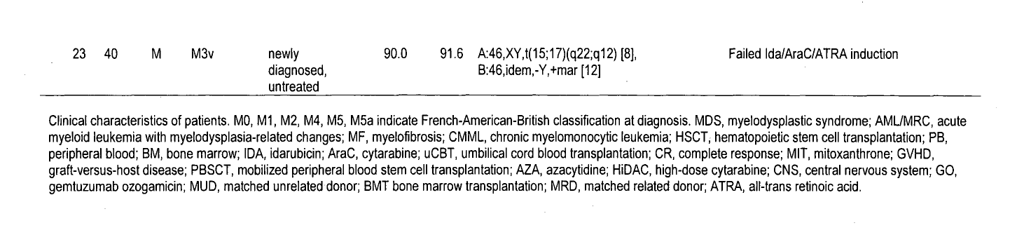

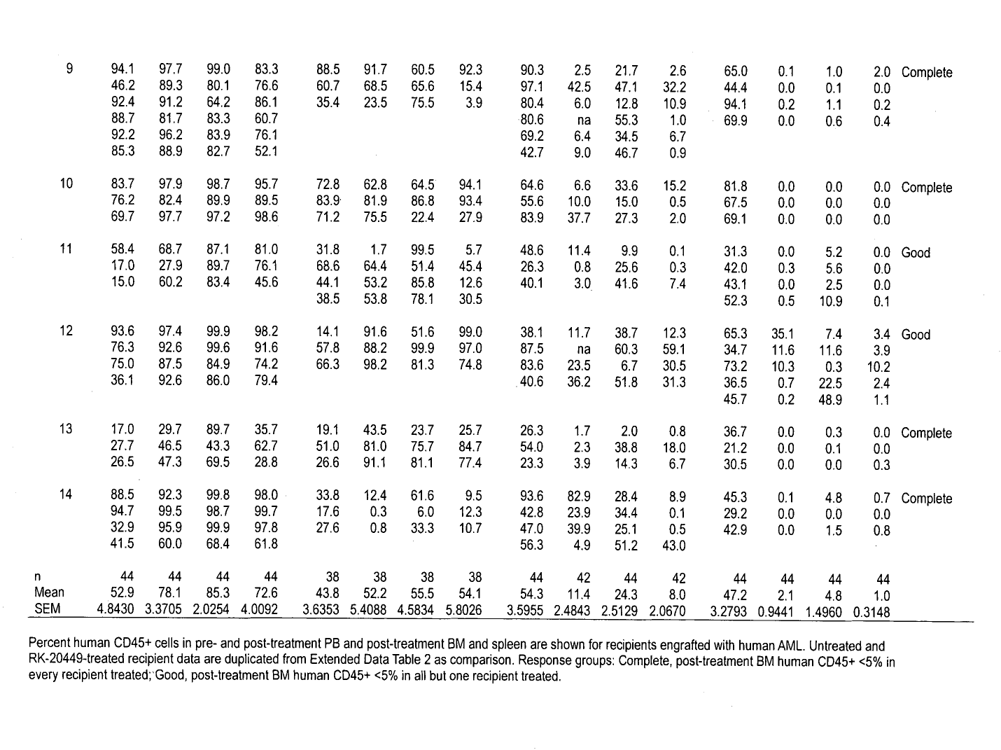

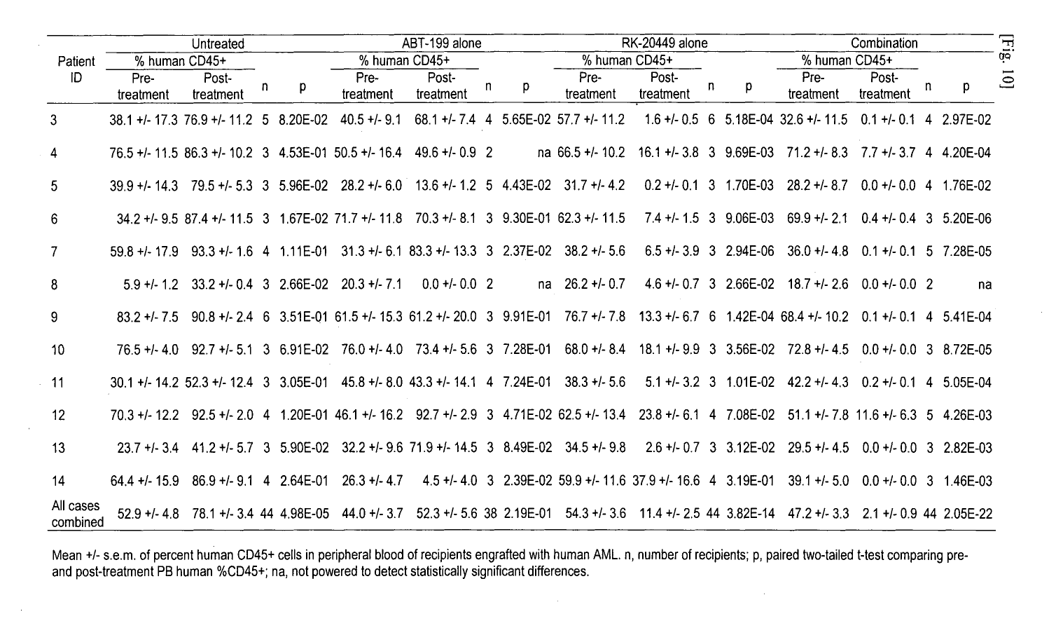

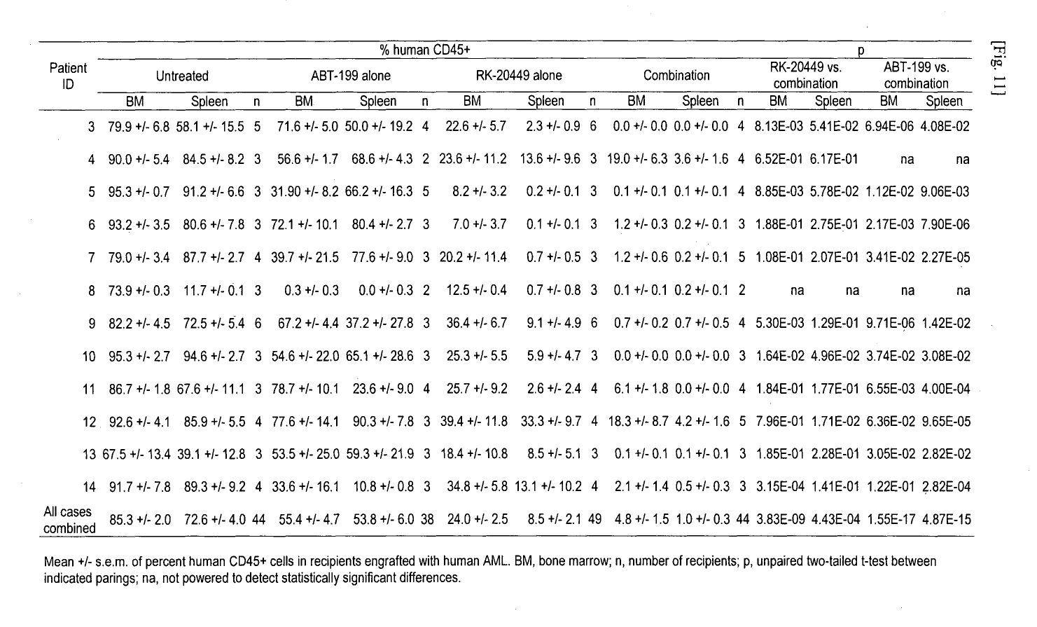

Recipients engrafted with AML from cases in which kinase inhibition alone did not completely eradicate human AML cells in the BM and spleen were treated with RK-20449 combined with ABT-199. See, FIGS. 9-11, for data and statistics. PB hCD45+AML cell chimerism time-course for treated recipients illustrate the differences in the rate and degree of peripheral responses associated with type of treatment (see, FIG. 6A). In the majority of cases, apoptosis induction by BCL-2 inhibitor ABT-199 alone resulted in limited responses. In contrast, in all 12 cases, combination treatment significantly reduced human AML chimerisms in PB, BM and spleen. Moreover, in 9 of 12 cases, combined inhibition of kinase and anti-apoptosis pathways led to elimination of human AML cells in vivo below the limit of detection without targeting other co-existing mutations (see, FIGS. 6B and 8).

Residual viable human AML cells were too few to assess functionally in most recipients that showed complete responses to RK-20449/ABT-199 combination treatment. To assess the effect of combination treatment to AML stem cells, human AML cells were isolated from cases that showed complete responses, but with relatively high residual BM human CD45 chimerisms, serial transplantation into NSG mice was performed (see, FIG. 6C). Leukemia-initiating capacity remaining after treatment was compared by isolating residual viable human CD45+AML cells from each treated mouse and transplanting a pre-determined fraction into secondary NSG recipients. The RK-20449 alone- and ABT-199 alone-treated BM contained enough viable LSCs to initiate AML in every secondary recipient transplanted. In contrast, among 23 mice transplanted with residual AML cells from recipients treated with combination treatment, only one engrafted, indicating that combination treatment with RK-20449 and ABT-199 more effectively reduced the frequency of LICs in vivo.

While the invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method of co-inhibiting HCK and BCL-2 in a cell, comprising contacting the cell with an HCK inhibitor and a BCL-2 inhibitor, or a pharmaceutically acceptable composition thereof.

2. A method of killing a cell having an FLT3−ITD mutation, comprising contacting the cell with an HCK inhibitor and a BCL-2 inhibitor, or a pharmaceutically acceptable composition thereof.

3. The method of claim 1 or 2, further comprising contacting the cell with an FLT3−ITD inhibitor.

4. The method of claim 1 or 2, wherein the HCK inhibitor is a dual HCK/FLT3−ITD inhibitor.

5. A method of treating acute myeloid leukemia, comprising conjointly administering to a subject an HCK inhibitor and a BCL-2 inhibitor, or a pharmaceutically acceptable composition thereof.

6. The method of claim 5, wherein the subject has FLT3−ITD+acute myeloid leukemia.

7. The method of claim 5 or 6, wherein the subject has malignant hematopoiesis and/or non-malignant multilineage hematopoiesis characterized by cells having one or more mutations in a gene selected from DNMT3A, IDH2, IDH1, NPM1, TET2, CEBPA, ASXL1, EZH2, SETBP1, SMC3, KIT, NRAS, and WT1.

8. The method of any one of claims 5-7, further comprising conjointly administering a FLT3−ITD inhibitor.

9. The method of any one of claims 5-8, wherein the HCK inhibitor is a dual HCK/FLT3−ITD inhibitor.

10. The method of claim 8 or 9, wherein the HCK inhibitor, the FLT3−ITD inhibitor, and the BCL-2 inhibitor are administered simultaneously or sequentially in separate unit dosage forms.

11. The method of any one of claims 5-10, comprising administering a single unit dosage form comprising an HCK inhibitor, a BCL-2 inhibitor, and a pharmaceutically acceptable carrier, adjuvant, or vehicle.

12. The method of claim 11, wherein the single unit dosage form further comprises an FLT3−ITD inhibitor, or wherein the HCK inhibitor is a dual HCK/FLT3−ITD inhibitor.

13. The method of any preceding claim, wherein the HCK inhibitor is selected from RK-20449, RK-20693, RK-24466, RK-20444, RK-20445, and RK-20466.

14. The method of any one of claim 3, 4, or 7-12, wherein the FLT3−ITD inhibitor is selected from AC220, sorafenib, PKC412, CEP-701, UNC2025, MLN518, KW-2449, AMG-925, sunitinib, SU5614, AC2206, crenolanib, and PLX3397.

15. The method of any preceding claim, wherein the BCL-2 inhibitor is selected from AT-101, TW-37, TM-1206, gossypolic acid, gossypolonic acid, apogossypol, apogossypolone, A385358, ABT-737, ABT-263, ABT-199, WEHI-539, BXI-61, BXI-72, obatoclax, JY-1-106, and SAHB peptides.

16. The method of claim 15, wherein the BCL-2 inhibitor is selected from gossypol, obatoclax, ABT-737, ABT-199, and ABT-263.

17. The method of claim 16, wherein the BCL-2 inhibitor is ABT-199.

18. The method of claim 17, wherein the HCK inhibitor is RK-20449 and the BCL-2 inhibitor is ABT-199.

19. The method of claim 17, wherein the HCK inhibitor is RK-20693 and the BCL-2 inhibitor is ABT-199.

20. The method of claim 17, wherein the FLT3−ITD inhibitor is AC220 and the BCL-2 inhibitor is ABT-199.

21. The method of claim 16, wherein the FLT3−ITD inhibitor is SU5614 and the BCL-2 inhibitor is ABT-737.

22. The method of any preceding claim, wherein the HCK inhibitor, and/or FLT3−ITD inhibitor, and/or the BCL-2 inhibitor is each present as a pharmaceutically acceptable salt.

23. The method of any preceding claim, wherein the HCK inhibitor, and/or FLT3−ITD inhibitor, and/or the BCL-2 inhibitor is each present in a pharmaceutically acceptable composition.

24. A composition for co-inhibiting HCK and BCL-2, comprising an HCK inhibitor and a BCL-2 inhibitor, or a pharmaceutically acceptable composition thereof.

25. A composition for killing a cell having an FLT3−ITD mutation, comprising an HCK inhibitor and a BCL-2 inhibitor, or a pharmaceutically acceptable composition thereof.

26. The composition of claim 24 or 25, further comprising contacting the cell with an FLT3−ITD inhibitor.

27. The composition of claim 24 or 25, wherein the HCK inhibitor is a dual HCK/FLT3−ITD inhibitor.

28. A composition for treating acute myeloid leukemia, comprising conjointly administering to a subject an HCK inhibitor and a BCL-2 inhibitor, or a pharmaceutically acceptable composition thereof.

29. The composition of claim 28, wherein the subject has FLT3−ITD+acute myeloid leukemia.

30. The composition of claim 28 or 29, wherein the subject has malignant hematopoiesis and/or non-malignant multilineage hematopoiesis characterized by cells having one or more mutations in a gene selected from DNMT3A, IDH2, IDH1, NPM1, TET2, CEBPA, ASXL1, EZH2, SETBP1, SMC3, KIT, NRAS, and WT1.

31. The composition of any one of claims 28-30, further comprising conjointly administering a FLT3−ITD inhibitor.

32. The composition of any one of claims 28-31, wherein the HCK inhibitor is a dual HCK/FLT3−ITD inhibitor.

33. The composition of claim 31 or 32, wherein the HCK inhibitor, the FLT3−ITD inhibitor, and the BCL-2 inhibitor are administered simultaneously or sequentially in separate unit dosage forms.

34. The composition of any one of claims 28-33, comprising administering a single unit dosage form comprising an HCK inhibitor, a BCL-2 inhibitor, and a pharmaceutically acceptable carrier, adjuvant, or vehicle.

35. The composition of claim 34, wherein the single unit dosage form further comprises an FLT3−ITD inhibitor, or wherein the HCK inhibitor is a dual HCK/FLT3−ITD inhibitor.

36. The composition of any preceding claim, wherein the HCK inhibitor is selected from RK-20449, RK-20693, RK-24466, RK-20444, RK-20445, and RK-20466.

37. The composition of any one of claim 26, 27, or 30-35, wherein the FLT3−ITD inhibitor is selected from AC220, sorafenib, PKC412, CEP-701, UNC2025, MLN518, KW-2449, AMG-925, sunitinib, SU5614, AC2206, crenolanib, and PLX3397.

38. The composition of any preceding claim, wherein the BCL-2 inhibitor is selected from AT-101, TW-37, TM-1206, gossypolic acid, gossypolonic acid, apogossypol, apogossypolone, A385358, ABT-737, ABT-263, ABT-199, WEHI-539, BXI-61, BXI-72, obatoclax, JY-1-106, and SAHB peptides.

39. The composition of claim 38, wherein the BCL-2 inhibitor is selected from gossypol, obatoclax, ABT-737, ABT-199, and ABT-263.

40. The composition of claim 39, wherein the BCL-2 inhibitor is ABT-199.

41. The composition of claim 40, wherein the HCK inhibitor is RK-20449 and the BCL-2 inhibitor is ABT-199.

42. The composition of claim 40, wherein the HCK inhibitor is RK-20693 and the BCL-2 inhibitor is ABT-199.

43. The composition of claim 40, wherein the FLT3−ITD inhibitor is AC220 and the BCL-2 inhibitor is ABT-199.

44. The composition of claim 39, wherein the FLT3−ITD inhibitor is SU5614 and the BCL-2 inhibitor is ABT-737.

45. The composition of any preceding claim, wherein the HCK inhibitor, and/or FLT3−ITD inhibitor, and/or the BCL-2 inhibitor is each present as a pharmaceutically acceptable salt.

46. The composition of any preceding claim, wherein the HCK inhibitor, and/or FLT3−ITD inhibitor, and/or the BCL-2 inhibitor is each present in a pharmaceutically acceptable composition.
